(19) United States
(12) Patent Application Publication
(10) Pub. No.: US 2026/0033808 A1
(43) Pub. Date: Feb. 5, 2026

(54) METHODS TO RECONSTRUCT 3D IMAGE/MAP FOR THE STIFFNESS OF SOFT TISSUES

(71) Applicants: North Carolina State University, Raleigh, NC (US); MAYO FOUNDATION FOR MEDICAL EDUCATION AND RESEARCH, Rochester, MN (US)

(72) Inventors: Murthy Guddati, Raleigh, NC (US); Abdelrahman Elmeliegy, Raleigh, NC (US); Matthew W. Urban, Rochester, MN (US)

(21) Appl. No.: 19/099,889

(22) PCT Filed: Jul. 30, 2023
(86) PCT No.: PCT/US2023/071296

§ 371 Date: Jan. 30, 2025

Related U.S. Application Data
(60) Provisional application No. 63/393,892, filed on Jul. 30, 2022.

(51) Int. Cl. A61B 8/00; G06T 7/00; G06T 15/00

(52) U.S. Cl. CPC A61B 8/485; A61B 8/5207; G06T 7/0012; G06T 15/00; G06T 2200/04; G06T 2207/10136; G06T 2207/30004; G06T 2210/41

(57) ABSTRACT

Various examples are provided related to methods and systems to reconstruct 3D image/map for the stiffness of soft tissues. In one example, a method includes obtaining particle velocities from generated mechanical waves in tissue of a subject; reconstructing a stiffness map of soft tissue in a region of interest (ROI) from the particle velocities; and generating an image of the stiffness map for rendering on a user device. In another example, a system includes an ultrasound scanner for shear wave elastography (SWE) and a computing or processing device that can obtain particle velocities; reconstruct a stiffness map of soft tissue in a ROI from the particle velocities; and generate an image of the stiffness map for rendering. The particle velocities can be generated from acoustic radiation forces produced by the ultrasound scanner.

### CROSS REFERENCE TO RELATED APPLICATIONS

This application claims priority to, and the benefit of, co-pending U.S. provisional application entitled “Method to Reconstruct 3D Image/Map for the Stiffness of Small Tissues” having Ser. No. 63/393,892, filed Jul. 30, 2022, which is hereby incorporated by reference in its entirety.

### STATEMENT REGARDING FEDERALLY SPONSORED RESEARCH OR DEVELOPMENT

This invention was made with government support under grant number HL 145268 awarded by the National Institutes of Health and under grant numbers CMMI1635291 and DMS2111234 awarded by the National Science Foundation. The government has certain rights in the invention.

### BACKGROUND

According to the World Health Organization (WHO), cancer is a leading cause of death worldwide, accounting for nearly 10 million deaths in 2020. Cancer mortality can be reduced if cases are detected and treated early. Soft tissue stiffness is an important biomarker in the diagnosis of cancerous tumors. From the pathology viewpoint, dense breast tissue is one of the most important factors for developing breast carcinoma. Magnetic Resonance Elastography (MRE), and ultrasound elastography are among the most advanced modalities that are used to provide a map of soft tissue stiffness (elasticity map, hence the name, elastography). Although MRE has been quite successful in this regard, ultrasound elastography started to gain more attention over the last decade owing to its shorter acquisition time, lower cost, portability and safety.

### SUMMARY

Aspects of the present disclosure are related to methods and systems to reconstruct 3D image/map for the stiffness of soft tissues. In one aspect, among others, a method comprises obtaining particle velocities from generated mechanical waves in tissue of a subject; reconstructing a stiffness map of soft tissue in a region of interest (ROI) from the particle velocities; and generating an image of the stiffness map for rendering on a user device. In one or more aspects, reconstructing the stiffness map of soft tissue in the ROI can be based upon a cross-correlation based cost functional. Reconstruction of the stiffness map can comprise minimizing the cross-correlation-based cost functional to within a defined threshold. Reconstruction of the stiffness map can comprise determining a back propagated misfit; and determining a gradient based upon the back propagated misfit and a forward propagating wavefield. Forward propagating wavefield and the back propagated misfit can be computed using full waveform modeling. The back propagated misfit can be a modified back propagated misfit.

In various aspects, the reconstruction can be carried out over a plurality of frequencies. One or more stiffness map from lower frequency data can be used as an initial estimate for refined stiffness maps from broader frequency data. The reconstruction can be carried out directly by matching time histories. In many aspects, the image of the stiffness map can be a three-dimensional (3D) image. The image of the stiffness map can be reconstructed from particle velocities on one or more lower dimensional manifold. The stiffness map can comprise elastic modulus variation or viscoelasticity variation. A viscosity map can be constructed after finalizing an elastic modulus map. A viscoelastic mechanical model can comprise a general frequency dependent complex modulus. The particle velocities can be generated from acoustic radiation forces. Multiple acoustic radiation forces can be used to increase sensitivity of measurements to viscoelasticity maps. The multiple acoustic radiation forces can be located on multiple sides of the ROI. Push locations can be based upon initial inspection of acoustic information. In some aspects, the ROI can be determined based upon initial inspection of acoustic information.

In another aspect, a system to reconstruct stiffness of soft tissue comprises: an ultrasound scanner configured for shear wave elastography (SWE); and a computing device comprising a processor and memory, the computing device configured to at least: obtain particle velocities using the ultrasound scanner, from generated mechanical waves in tissue of a subject; reconstruct a stiffness map of soft tissue in a region of interest (ROI) from the particle velocities; and generate an image of the stiffness map for rendering on the computing device or a user device. In one or more aspects, reconstructing the stiffness map of soft tissue in the ROI can be based upon a cross-correlation based cost functional. Reconstruction of the stiffness map can comprise minimizing the cross-correlation-based cost functional to within a defined threshold. Reconstruction of the stiffness map can comprise determining a back propagated misfit; and determining a gradient based upon the back propagated misfit and a forward propagating wavefield. The forward propagating wavefield and the back propagated misfit can be computed using full waveform modeling. The back propagated misfit can be a modified back propagated misfit.

In various aspects, the reconstruction can be carried out over a plurality of frequencies. One or more stiffness map from lower frequency data can be used as an initial estimate for refined stiffness maps from broader frequency data. The reconstruction can be carried out directly by matching time histories. In many aspects, the image of the stiffness map can be a three-dimensional (3D) image. The image of the stiffness map can be reconstructed from particle velocities on one or more lower dimensional manifold. The stiffness map can comprise elastic modulus variation or viscoelasticity variation. A viscosity map can be constructed after finalizing an elastic modulus map. A viscoelastic mechanical model can comprise a general frequency dependent complex modulus. The particle velocities can be generated from acoustic radiation forces produced by the ultrasound scanner. Multiple acoustic radiation forces can be used to increase sensitivity of measurements to viscoelasticity maps. The multiple acoustic radiation forces can be located on multiple sides of the ROI. Push locations can be based upon initial inspection of acoustic information. In some aspects, the ROI can be determined based upon initial inspection of acoustic information.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 illustrates an example of a two-dimensional (2D) shear wave elastography (SWE) setup and default Acoustic Radiation Force (ARF), in accordance with various embodiments of the present disclosure.

FIG. 2 illustrates a comparison of normalized wavefields for different mesh resolution, in accordance with various embodiments of the present disclosure.

FIG. 3 illustrates an example of convergence behavior of L₂ and CC cost functionals, in accordance with various embodiments of the present disclosure.

FIG. 4 illustrates an example of comparison between L₂ functional and proposed correlation functional, in accordance with various embodiments of the present disclosure.

FIG. 5 illustrates examples of inclusion shapes, in accordance with various embodiments of the present disclosure.

FIG. 6 illustrates an example of nonlinearity of cost functional for different inclusion shapes when using CC and L₂ functionals, in accordance with various embodiments of the present disclosure.

FIG. 7 illustrates an example of a modified ARF configuration, in accordance with various embodiments of the present disclosure.

FIG. 8 illustrates examples of convergence behavior of two-parameter inversion without and with noise, in accordance with various embodiments of the present disclosure.

FIGS. 9-12 illustrate examples of reconstructed images of shear wave velocity distribution without and with noise, in accordance with various embodiments of the present disclosure.

FIG. 13 illustrates examples of convergence behavior for inverting an entire ROI and just an inclusion region with noise, in accordance with various embodiments of the present disclosure.

FIGS. 14A and 14B illustrate an example of the ultrasound imaging setup and proposed reconstruction in the x-y plane, in accordance with various embodiments of the present disclosure.

FIGS. 15-17 illustrate examples of shear elasticity (modulus) and viscosity maps, in accordance with various embodiments of the present disclosure.

FIGS. 18 and 19 illustrate examples of reconstruction of tumor-like tissues, in accordance with various embodiments of the present disclosure.

FIGS. 20A and 20B illustrate examples of pushes and acquisition planes, in accordance with various embodiments of the present disclosure.

FIGS. 21 and 22 illustrate examples of reconstruction with two and four push configurations, in accordance with various embodiments of the present disclosure.

FIG. 23 illustrates an example of reconstruction of viscosity after fixing shear modulus, in accordance with various embodiments of the present disclosure.

FIGS. 24A and 24B illustrate the configuration of a cylinder relative to SWE acquisition and projected on a 2D plane (x-y), in accordance with various embodiments of the present disclosure.

FIG. 25 illustrates an example of shear wave propagation in phantom, in accordance with various embodiments of the present disclosure.

FIGS. 26-28 illustrate examples of B-mode images of phantoms, in accordance with various embodiments of the present disclosure.

FIGS. 29 and 30 illustrate examples of shear wave motions measured on a central plane of phantoms, in accordance with various embodiments of the present disclosure.

FIGS. 31 and 32 illustrate examples of Fourier transformed data for phantoms, in accordance with various embodiments of the present disclosure.

FIG. 33 illustrates an example of push sequence and timing followed in SW generation and acquisition, in accordance with various embodiments of the present disclosure.

FIGS. 34-41 illustrate examples of comparisons between the simulated and measured shear wave motion in phantoms, in accordance with various embodiments of the present disclosure.

FIGS. 42-45 illustrate examples of reconstructed shear modulus maps for phantoms, in accordance with various embodiments of the present disclosure.

FIG. 46 illustrates an example of an ultrasound shear wave imaging setup, in accordance with various embodiments of the present disclosure.

FIGS. 47A and 47B illustrate examples of true effective shear modulus distribution for tumors of different diameters, in accordance with various embodiments of the present disclosure.

FIGS. 48A and 48B illustrate examples of reconstructed 3D maps for a tumor at different resolutions, in accordance with various embodiments of the present disclosure.

FIGS. 49 and 50 illustrate examples of reconstructed 2D views of effective shear modulus and comparison between the reconstructed and true effective shear modulus, in accordance with various embodiments of the present disclosure.

FIGS. 51A and 51B illustrate examples of reconstructed 3D maps for a tumor at different resolutions, in accordance with various embodiments of the present disclosure.

FIGS. 52 and 53 illustrate examples of reconstructed 2D views of effective shear modulus and comparison between the reconstructed and true effective shear modulus, in accordance with various embodiments of the present disclosure.

FIGS. 54A and 54B illustrate examples of reconstructed 3D maps for a tumor at different resolutions, in accordance with various embodiments of the present disclosure.

FIGS. 55 and 56 illustrate examples of reconstructed 2D views of effective shear modulus and comparison between the reconstructed and true effective shear modulus, in accordance with various embodiments of the present disclosure.

FIGS. 57A and 57B illustrate examples of reconstructed 3D maps for a tumor at different resolutions, in accordance with various embodiments of the present disclosure.

FIGS. 58 and 59 illustrate examples of reconstructed 2D views of effective shear modulus and comparison between the reconstructed and true effective shear modulus, in accordance with various embodiments of the present disclosure.

FIG. 60 is a schematic block diagram illustrating an example of a system employed for estimation of stiffness, in accordance with various embodiments of the present disclosure.

### DETAILED DESCRIPTION

Disclosed herein are various examples related to methods and systems to reconstruct 3D image/map for the stiffness of soft tissues. Reference will now be made in detail to the description of the embodiments as illustrated in the drawings, wherein like reference numbers indicate like parts throughout the several views.

Magnetic resonance imaging MRI and Ultrasound imaging are among the most advanced modalities that are widely used to provide a map of soft tissue stiffness. Although the success that MRI has been achieved in that regard, ultrasound imaging started to gain more attention over the last decade owing to its shorter acquisition time, lower cost, portability, and more importantly it is safer to be applied to all patients. While there exist many methods to provide 2D stiffness maps of soft tissues, these methods suffer from several technical limitations, are more prone to erroneous reconstruction, and cannot provide a 3D stiffness map of soft tissues using the available ultrasound transducers.

Shear wave elastography is a method for the reconstruction of the stiffness of soft tissues such as tumors, by matching the measured and the simulated wave fields generated by Acoustic Radiation Force (ARF) using an inverse optimization scheme. Currently, all of the commercial reconstruction schemes are based on local inversion methods. The main idea of the local inversion method is based on an idealization of the wave propagation in the soft tissues; specifically, it assumes local homogeneity and plane wave propagation. Time-of-flight (TOF), and direct wave equation inversion methods, among others, are examples of the local inversion approaches. These idealizations make those methods prone to erroneous reconstruction due to loss of information associated with reflections, refractions including wave bending.

Another family of inversion is based on global inversion methods, where the above limitation is overcome by a better description of the wave propagation in the soft tissue, which includes full scattering and full heterogeneity modeling, e.g., using Full-Waveform Inversion (FWI). However, in addition to the high computational cost of FWI, one of the main bottlenecks is that FWI is a highly nonlinear problem that is challenging to solve. In addition, all of the current reconstruction methods can only provide a 2D stiffness map given wavefield data on a single plane. This is because ultrasound transducers can only detect wavefield on a single plane at a time with acceptable accuracy. Moreover, the current reconstruction methods are based on minimizing a cost functional that is based on the ordinary least-squares norm of the errors. These cost functionals have shown high nonlinearity, hence slow convergence and/or erroneous reconstruction, especially when the data are scarce.

The disclosed methodology can reconstruct 3D stiffness maps of soft tissues given wave fields' data inside the soft tissues on a plane. The methodology comprises data processing, which includes: (a) a better description of the wave propagating in the soft tissues, including complex heterogeneous mediums, (b) use of 2D measurements to construct 3D stiffness map, and (c) a computationally efficient reconstruction method that is based on a cross-correlation-based cost functional, enabling faster and convergent reconstruction method. The new method has been verified using noise-laden synthetic data in 2D, which can be extended to 3D. Finally, the method will be validated using phantom experiment data provided by Mayo Clinic.

The methodology is the first to use cross-correlation-based cost functional in the reconstruction method as opposed to the widely used ordinary least squares-based functionals, enabling a more robust reconstruction algorithm. It is also the first to use 1D measurements to reconstruct 2D stiffness maps of soft tissues, which would mitigate the available apparatus limitations. This can be beneficial in the context of 3D tumor imaging, where the objective is to obtain the 3D image from 2D measurements. These two ideas offer a new framework for tumor imaging utilizing shear waves which that can result in a paradigm shift in the shear wave elastography field.

This methodology can be used to perform several types of ultrasound imaging used for providing stiffness maps of soft tissues. This includes tumors in any organ including but not limited to, breast, liver, and prostate. The methodology can be executed using a device comprising computing or processing circuitry (e.g., comprising a processor and memory) configured to process acoustic information as disclosed. This technology can augment many existing shear wave elastography hardware, which can handle acoustic radiation force and associated shear wave elastography data acquisition.

### Correlation-Based Full-Waveform Shear Wave Elastography

Generally, there are two main elastography techniques, static and dynamic. In static elastography, Hook's law-based inversion is used to estimate the elastic modulus from the measured displacement field. However, because the boundary conditions between different tissues are not precisely known, these techniques cannot quantitatively assess the mechanical properties of soft tissues. On the other hand, dynamic elastography, e.g., shear wave elastography (SWE), can provide a quantitative elasticity map with better contrast, by exciting the tissue with a shear wave that is generated by focusing acoustic beams inside the tissue (the resulting push is referred to as Acoustic Radiation Force, ARF). These shear waves are then tracked by an ultrafast ultrasound scanner which tracks the wavefield over the entire region of interest (ROI) with high accuracy.

Over the last decade, several dynamic reconstruction techniques have been developed which can be classified into two main categories, local and global. Some examples of local reconstruction techniques are, direct inversion of wave equation, Time-of-Flight (TOF), Plane Wave Elastography (PWE), and phase velocity based inversion. Direct wave equation inversion is straightforward but requires the wavefield to be known in the entire ROI. Furthermore, direct inversion of wave equation results in an operator that is highly sensitive to noise. These drawbacks make direct inversion not a reliable imaging technique. On the other hand, TOF, PWE and phase velocity-based methods assume that waves propagate with plane wavefronts. In addition, the medium is assumed to be locally homogeneous and have no scattered waves. For instance, TOF constructs a map of the shear wave speed after calculating the time delays for all spatial points in the direction of the wave propagation. This method is most used in commercial scanners in different contexts, e.g., Fast Shear Compounding, Supersonic Shear Imaging, and Comb-Push Ultrasound Shear Elastography.

Global (dynamic) reconstruction techniques, e.g., Full-Waveform Inversion (FWI), alleviate the disadvantages of the local reconstruction techniques by more accurately capturing the wave propagation inside the tissue, i.e., no assumption of wave propagation is made and all types of wave phenomena, including refractions, reflections and scattering associated with heterogeneities are accurately captured. Another attractive feature of the global reconstruction approaches is that noise can be readily handled. However, FWI is computationally expensive and, more importantly, it is highly nonlinear potentially leading to erroneous reconstructions. Moreover, the push amplitude is not precisely known, which may result in failure of accurate reconstruction. Other global techniques such as semi-analytical approaches provide good results with less computational cost but are limited to simple geometries and cannot provide elasticity map with arbitrary heterogeneities.

The choice of the cost functional and the parameterization are two main factors that affect the robustness of any reconstruction algorithm. In contrast to the conventional least-squares (L₂) cost functional, the cross-correlation (CC) based cost functional emphasizes the phase information by measuring the zero-lag cross-correlation between the measured and the simulated wavefields, rendering the inversion insensitive to the push amplitude. In addition, CC functionals are less nonlinear, hence less sensitive to the initial guess. Several studies in the geophysics field have demonstrated the success of the CC functionals.

In light of the above discussion, in this work, the issues associated with the FWI for ultrasound elastography are addressed and a CC cost functional proposed, that leads to reducing the nonlinearity of the FWI problem and results in a reconstruction algorithm that is independent of push amplitudes, less sensitive to the initial guess, and has a better convergence behavior compared to the classical L₂ cost functional. Furthermore, conventional ultrasound transducer measured a component of the wavefield in a single plane, but an objective is to map a 3D tumor. Motivated by this, the reconstruction of 2D elastography images from measurements on a single line is a consideration. Building on this proof-of-concept investigation, one goal is to develop 3D imaging of tumors using ultrasound measurements using existing ultrasound transducers.

METHODS. The basic approach of the reconstruction method is to iteratively modify the elasticity map until the simulated particle velocities match well with the measured particle velocities. Two aspects of this process are (a) the forward model, which provides the simulated responses for any given estimates of elastic properties, and (b) the cost functional, which encapsulates the goodness of the match between measured and simulated responses. In this section the forward model, which is essentially governed by a two-dimensional scalar wave equation, is first reviewed. In later subsections the cost functional, first reviewing the common least-squares approach and followed by the presentation of the proposed CC cost functional, is examined. Also presented is the summary of the gradient optimization algorithm utilized in the study, along with the derivation of appropriate gradients that are central to the algorithm. Finally, the overall setup for the in-silico experiments is described.

Forward model. In this work, consider Ω_{ROI}∈ [symbol] ⁿ to denote the region of interest (ROI) in soft tissues. The methodology is general and is expected to extend to 3D as well, but it initially focuses on 2D problems in this study, i.e., n=2 in the present context. Further, assume that the medium is isotropic and linearly elastic (and ignore any viscous effect at this time). The assumption of linearity follows that Acoustic Radiation Force (ARF) is very low in amplitude that the nonlinear effects do not enter into picture. Moreover, consider the elastic modulus to not vary within the observation time, e.g., ignore elastic modulus changes due to interstitial fluid pressure changes associated with poroelasticity. Finally, focus on the scenario with ARF applied inside the ROI, generating shear waves that travel through the ROI. Given these assumptions, the shear wave propagation inside the ROI can be represented using the scalar wave equation:

$$- ∇ . ( μ ( x ) ∇ u ( x , t ) ) + ρ \ddot{u} ( x , t ) = f ( x , t ) , x ∈ {Ω}_{ROI} \qquad ( 1.1 )$$

where u(x, t) is the wavefield, μ(x) is the shear modulus of the tissue, ρ is the tissue density and f(x, t) is the ARF push function. Further simplification can be applied by noticing that,

$$c ( x ) = \sqrt{\frac{μ ( x )}{ρ}} \qquad ( 1.2 )$$

where c(x) is the shear wave speed inside the soft tissue. This results in,

$$- ∇ . ( μ ( x ) ∇ u ( x , t ) ) + ρ \ddot{u} ( x , t ) = f ( x , t ) , x ∈ {Ω}_{ROI} \qquad ( 1.3 )$$

where explicitly showing the dependency on time and space is omitted for convenience.

Here, Equation (1.3) can be solved in the frequency domain to provide better control on the numerical dispersion especially at higher frequencies, and to facilitate parallelization over frequencies, leading to a more computationally efficient reconstruction. Thus, Fourier transform of Equation (1.3) in time, results in the reduced wave equation in frequency (ω) domain,

$$- ∇ \cdot ( {c}^{2} ∇ 𝒰 ) - {ω}^{2} = \qquad ( 1.4 )$$

where [symbol] and [symbol] are respectively the wavefield and the forcing function in (x, ω) space (Fourier transforms of u and f). Most of the soft tissue is viscous, thus the wave damps out as it moves away from the ARF. Also, assuming that the tumor is small relative to the organ, and it exists relatively far from the surface. Given these observations, assume that the background medium is a homogenous medium around the tumor and the waves travel outside the ROI without being reflected back into the ROI. Therefore, the radiation condition on the boundary can be utilized. The boundary value problem can be stated as follows: given c, ω and [symbol] , find [symbol] such that,

$$- ∇ \cdot ( {c}^{2} ∇ 𝒰 ) - {ω}^{2} = , x ∈ {Ω}_{ROI} ∈ {ℝ}^{2} , ∇ 𝒰 \cdot n = Λ𝒰 , x ∈ Γ ≡ \partial Ω \qquad ( 1.5 )$$

where n is the outward normal and Λ is the Dirichlet-to-Neumann operator representing the radiation boundary condition, often approximated by an absorbing boundary condition.

Equation (1.5) is typically solved using a discretization-based method such as finite element or finite difference methods. The discretized version of the scalar wave Equation (1.5) can then be written as,

$$= b \qquad ( 1.6 )$$

where [symbol] is the discrete wave operator obtained using, e.g., finite element or finite difference methods, [symbol] and b are respectively the nodal displacements (wavefield) and force vectors (ARF push). In this work, the standard finite element method can be utilized with 2D bilinear finite elements (4-node bilinear element) for modeling the physical ROI described in Equation (1.5), leading to finite (N_{dofs}) degree of freedoms (DOFs). In addition, the absorbing boundary condition in Equation (1.5) is modeled using Perfectly Matched Discrete Layers which is a discrete version of the Perfectly Matched Layers. Typically, 3-5 PMDL layers are sufficient to absorb the waves efficiently, leading to additional m_{pmdl} DOFs. This results in a linear system of equations represented by Equation (1.6), where the size of matrix [symbol] is (N_{dofs}+m_{pmdl})×(N_{dofs}+m_{pmdl}), and the size of vectors [symbol] and b is (N_{dofs}+m_{pmdl}).

Conventional reconstruction approaches. In this section the reconstruction method based on the adjoint-based gradient optimization, which minimizes the misfit between the measured and predicted responses in a least-squares sense, is reviewed. The objective of the reconstruction is to obtain the distribution of the shear wave speed in the ROI. The discretized version of the shear wave speed distribution takes the vector form, which is the model parameter vector ( [symbol] ) of size m (m is related to the spatial resolution of the reconstruction; higher the resolution, bigger the value of m). The goal of reconstruction is to find the optimizing model parameter vector [symbol] , i.e., optimal shear wave velocity distribution, by minimizing a cost functional [symbol] that captures the misfit between the measured and predicted responses:

$${\hat{}}^{} = argmin ( ) . \qquad ( 1.7 )$$

A conventional choice of the cost functional is related to least-squares minimization of the misfit, which is described below, along with its gradient, which is a quantity used in many optimization methods.

Least-squares cost functional and its gradient. Let d in Ω_{data}⊆Ω_{ROI} and [symbol] be the measured and the predicted wavefields, respectively. The least-squares cost functional is defined as,

$$( 𝓂 ) = \frac{1}{2} {( ( ) - 𝒹 )}^{†} ( ( ) - 𝒹 ) \qquad ( 1.8 )$$

where [symbol] is an observation operator, which is a projection matrix that restricts the full wavefield [symbol] to the measurement locations, and † represents conjugate transpose.

The goal of the reconstruction is to find the minimizer of the cost functional. To this end, we follow the standard gradient optimization approach; we summarize the basic idea here and the reader is referred to (Tarantola, 1984) for further details: We start with Taylor series expansion of Equation (1.8) in the vicinity of a starting point [symbol] :

$$( {0}_{} + Δ ) \approx ( {0}_{} ) + Δ {T}^{} + \frac{1}{2} Δ {T}^{} Δ \qquad ( 1.9 )$$

where [symbol] is the gradient vector, [symbol] is the Hessian matrix and T represents matrix transposition. Assuming that the Hessian is invertible, the estimate of the perturbation Δ [symbol] which minimizes the functional in Equation (1.9) is determined by

$$Δ = - {- 1}^{} . \qquad ( 1.1 )$$

For a small number of model parameters m, the finite difference approximation can be utilized for the gradient computation (by perturbing each parameter). However, when m is large, taking finite differences by perturbing each parameter may be highly inefficient. Instead, the adjoint formulation for the gradient calculation can be utilized where the i^{th} component of the gradient [symbol] can be expressed as,

$${i}_{} = = \{ {†}^{} v \} , \qquad ( 1.11 )$$

$$i = 1 , … , m ,$$

where v is the adjoint variable which can be obtained by solving the adjoint equation:

$${†}^{} v = {T}^{} ( 𝒹 - ) \qquad ( 1.12 )$$

where d− [symbol] is the misfit computed at the measurement's location, and [symbol] ^{T} is an expansion operator that has one as entries corresponds to the measurements and zeros otherwise. ( [symbol] ^{†})⁻¹ [symbol] ^{T}( [symbol] − [symbol] ) can be interpreted as a backpropagation of the misfit to the entire computational domain.

Gradient-based reconstruction requires an initial model to start the iterations. If the initial model is significantly different from the ground truth, the optimization may converge to an incorrect solution, since the L₂ cost functional may have many local minima. The situation with imaging becomes worse at higher frequencies due to the phenomena of cycle skipping. In addition, if the push amplitude is not precisely known, the iterative minimization may lead to a local minimum which may be far away from the global minimum, leading to erroneous reconstruction and the need for a method that can mitigate all or some of these challenges.

Proposed Reconstruction Methodology. In this section, the cross-correlation (CC) based functional is presented as an alternative to the classical L₂-norm of the misfit. Such functionals have been used in geophysical inversion with better success compared to the L₂-norm of the misfit. Essentially, CC functional captures the correlation between the measured and the predicted wavefields by computing the cross-correlation of the measured and the predicted wavefields. Hence, the cost functional is independent of the push amplitudes which is often not precisely known. More importantly, CC functionals emphasize the phase information as opposed to amplitude mismatch leading to a significantly better behaving algorithm with no additional cost. In what follows, the CC functional is presented and its gradient derived based on the adjoint formulation.

Cross-Correlation (CC) cost functional. A CC cost functional [symbol] can be used that measures the (zero-lag) correlation between the two signals [symbol] and [symbol] , which can be defined as follows,

$$= 1 - \frac{{\vert 𝒹 . ( ) \vert}^{2}}{{( ‖ 𝒹 ‖ ‖ ‖ )}^{2}} = 1 - \frac{{( )}^{†} 𝒹 {𝒹}^{†} ( )}{{𝒹}^{†} {𝒹 ( )}^{†} ( )} . \qquad ( 1.13 )$$

The second term is essentially the square of the normalized cross-correlation. The term is negated to convert the problem to a minimization problem. Note that squaring of the cross-correlation is introduced as it appears to increase the convexity of the cost functional. It also facilitates cleaner derivation of the gradient, which is presented below.

Gradient of CC Functional. Using the adjoint formulation, the gradient can be written as,

$${i}_{} = \{ α ) + {( α ( ) )}^{†} - β ( ℓ + {( ℓ )}^{†} ) \} \qquad ( 1.14 )$$

which can be simplified as,

$${i}_{} = 2 \{ α ( ) - β ( ℓ ) \} \qquad ( 1.15 )$$

$$α = \frac{1 -}{{( )}^{†} 𝒹} , β = \frac{1 -}{{( )}^{†} ( )} ,$$

$$= {( {†}^{} )}^{- 1} {}^{} 𝒹 , ℓ = {( {†}^{} )}^{- 1}$$

This can be further simplified as,

$${i}_{} = 2 \{ ( - β ℓ ) \} = 2 \{ {†}^{} \} \qquad ( 1.16 )$$

Where [symbol] is given by:

$$= {( {†}^{} )}^{- 1} ( α 𝒹 - β ) . \qquad ( 1.17 )$$

Comparing the adjoint in Equations (1.17) and (1.12), the right hand side in Equation (1.12) (misfit) is modified by introducing α and β. Intuitively, α and β can be interpreted as shifting and weighing factors; when α=β=0.5, we get back Equation (1.12). Essentially comparing Equations (1.17) and (1.12) indicates that the computational cost of adjoint, and thus gradient computation is essentially identical between traditional least-squares and proposed CC functional.

Reconstruction Algorithm. Given that the cost functional is defined and the associated gradients are derived, a standard gradient-based optimization framework can be utilized to perform the reconstruction. Based on prior experience with seismic imaging problems, a BFGS algorithm was utilized, which turned out to perform well for the numerical examples explored in this work. There are other gradient-based optimization algorithms that can be used to replace the BFGS method, but the BFGS method is an effective choice for material inversion and other methods were not explored given the success with the BFGS method. The steps are presented in Algorithm 1.1.

Algorithm 1.1: Proposed reconstruction algorithm based on CC cost functional.

```
  - Given the measurements d.
  - Discretize the ROI into small pixels representing the shear modulus spatial
    distribution m₀.
  - Given an initial model m₀ and stopping criterion e.g. ∥g∥ ≤ ε, minimize the cost
    functional in Equation (1.13) over all frequencies and pushes until convergence.
    Each iteration k of the minimization process involves the following steps:
1. Obtain the preliminary step Δmₖ in Equation (1.10) using the following steps:
  a. Compute the modified back propagated misfit [symbol]     in Equation (1.17). This requires
    one adjoint solve.
  b. Compute the gradient gₖ using Equation (1.16).
  c. Solve Equation (1.10) using the BFGS algorithm to get the step Δmₖ.
2. Using cubic line search, minimize the cost function in Equation (1.13) to compute the
    step size α.
3. Update the model:
  a. \hat{m}=mₖ + αₖΔmₖ.
4. Repeat step 1 until ∥gₖ∥ < ε.
```

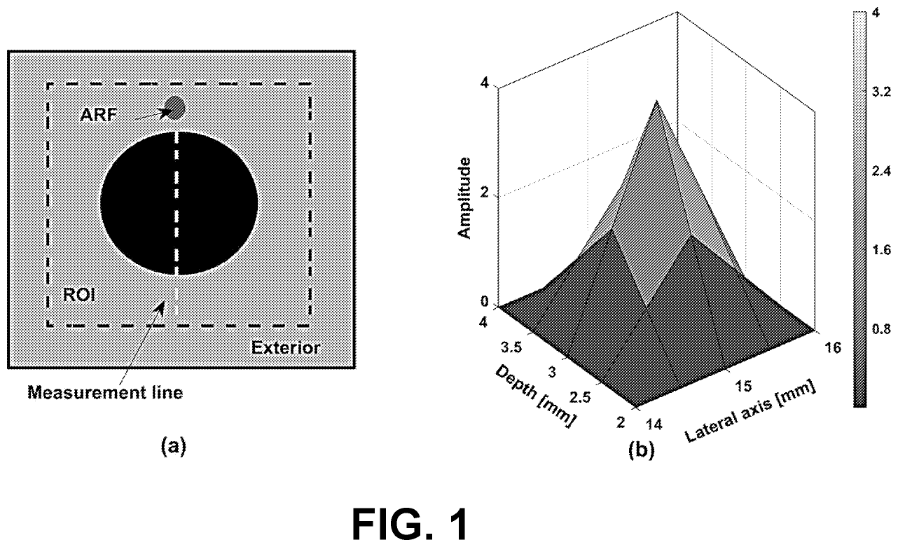

In Silico Experimental Setup. Starting with the observation that conventional ultrasound scanners measure (single component of) the particle velocity in a single plane, whereas the tumors to be imaged are 3D in nature. This reality can be encapsulated in a simpler setting, where the goal is to obtain a 2D image given the measurement on a single line. FIG. 1 is a schematic of a two-dimensional SWE setup illustrating: (a) the default setup and (b) the default ARF configuration. The expectation is that if a method is effective in the proposed 2D setting, it could be extended to realistic imaging of 3D tumors using SWE data. Results will be presented using in-silico experiments to illustrate the performance of the proposed method in two different scenarios of SWE, first in an idealized setting and then in more realistic settings. In this section however, the overall setup for the in-silico experiments is described, borrowing from past work with any deviations from the general setup mentioned in each of the specific experiments.

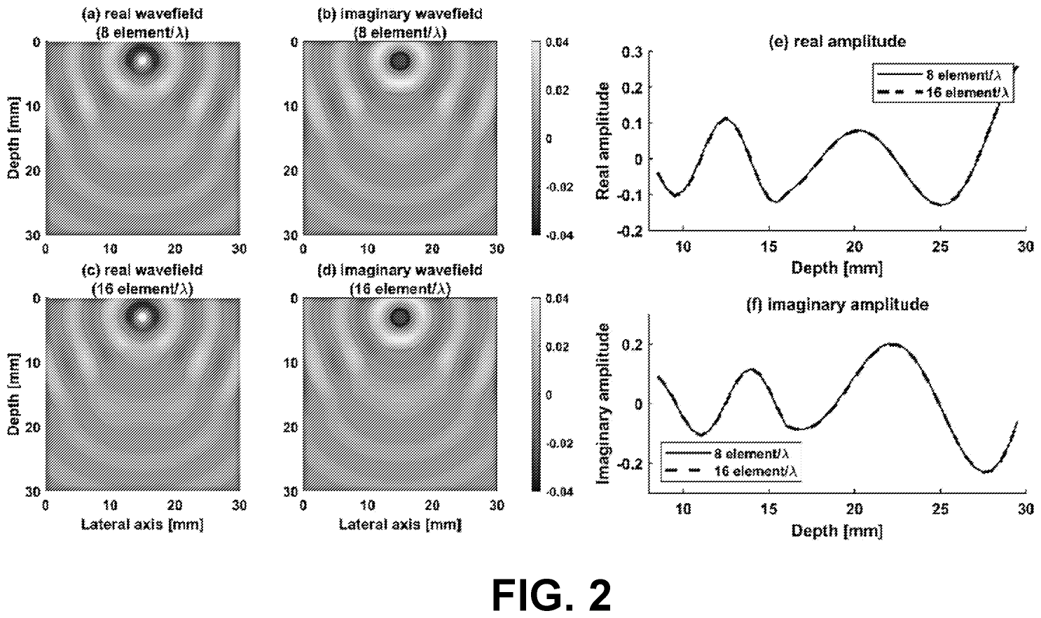

The Region of Interest (ROI): The default ROI is taken as 30 mm×30 mm square. The inclusion shape can vary but its nominal diameter is 14 mm. The shear wave velocity of the background and the inclusion are 2.88 and 5.16 m/sec, respectively. As mentioned earlier, the forward model was solved in the frequency domain using the finite element method. Following a 60×60 finite elements mesh, which guarantees at least eight elements per wavelength. This is consistent with the widely used protocol for wave simulations. FIG. 2 illustrates a comparison of normalized wavefields at 500 Hz for different mesh resolution. Plots (a) and (b) show the real and imaginary parts of responses using 8 elements per wavelength. Plots (c) and (d) show the real and imaginary part using 16 elements per wavelength. Plots (e) and (f) show a comparison of real and imaginary parts of the wavefield sampled on the vertical line at the middle of the ROI.

Acoustic Radiation Force (ARF): In in vivo settings, the problem including the ARF is 3D in nature and can be accurately modeled with the help of acoustic simulation. Since the idealization is in 2D, the ARF push can be approximated to be (discretized) sine squared pulse in space. Unless otherwise mentioned, the default ARF configuration in FIG. 1 can be used when generating the synthetic and predicted wavefields. It will be demonstrated that the proposed method is not sensitive to the ARF push configuration.

Choice of frequency content: In this study, consider different frequency contents depending on the complexity of the reconstruction. The frequency content can be chosen so that there are a wide range of wavelengths contributing to the reconstruction, thus leading to proper spatial resolution to image the tumor (as the method is to be applied to heterogeneous inclusions). For instance, the lowest frequency can be chosen so that the longest wavelength is contained in the ROI. On the other hand, the maximum frequency can be chosen so that it can resolve the smallest object to be imaged (better resolution). Similarly, the frequency increment can be as big as 100 Hz when reconstructing handful parameters, and as small as 10 Hz when reconstructing hundreds of parameters.

Inversion parameters: Start with assuming that the inclusion is homogenous, and the background modulus is known, and only reconstruct inclusion shear wave speed (one parameter). Then, assume that the background modulus is also unknown but homogeneous and reconstruct both the shear wave speeds of the background and inclusion (two parameters). Finally, the most complicated scenario is presented where it is assumed that the entire ROI is heterogenous and reconstruct the shear wave speed distribution (several hundreds of parameters).

RESULTS. Preliminary Comparison with Least-squares Functional. Before presenting the results using realistic in-silico SWE experiments, the main differences between the performance with L₂ and CC functionals are illustrated using a hypothetical experiment, where a horizontally propagating plane (shear) wave is scattered by an inclusion of diameter 20 mm, which has an analytical solution. The shear moduli of the background and the inclusion are 12 and 36 kPa, respectively. Consider the measurement strictly inside the inclusion, on the vertical diameter sampled every 1 mm, leading to a total of 18 data points. The initial estimate of the inclusion modulus is assumed to be the same as the background modulus of 12 kPa.

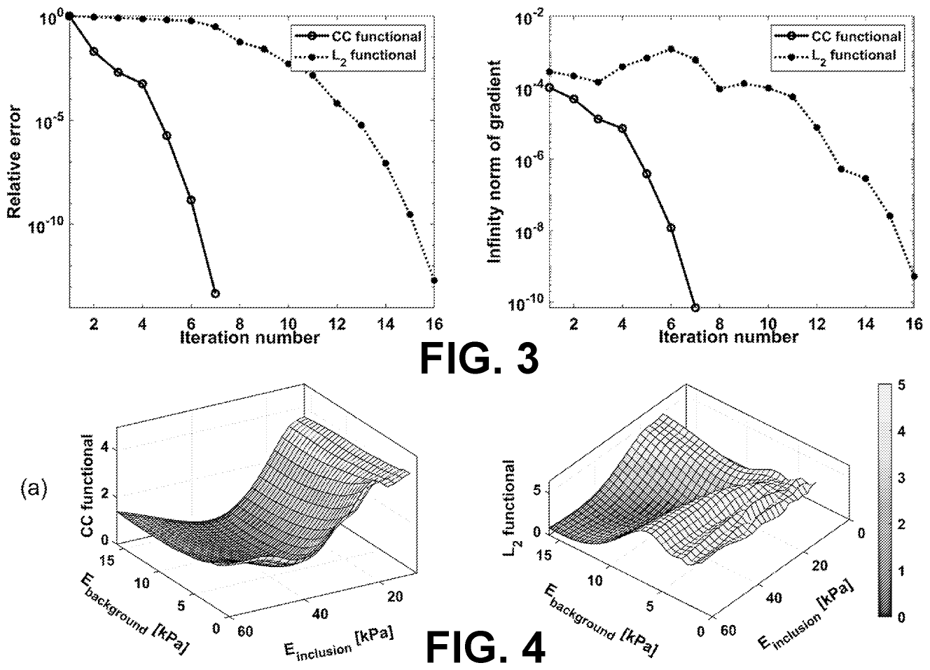

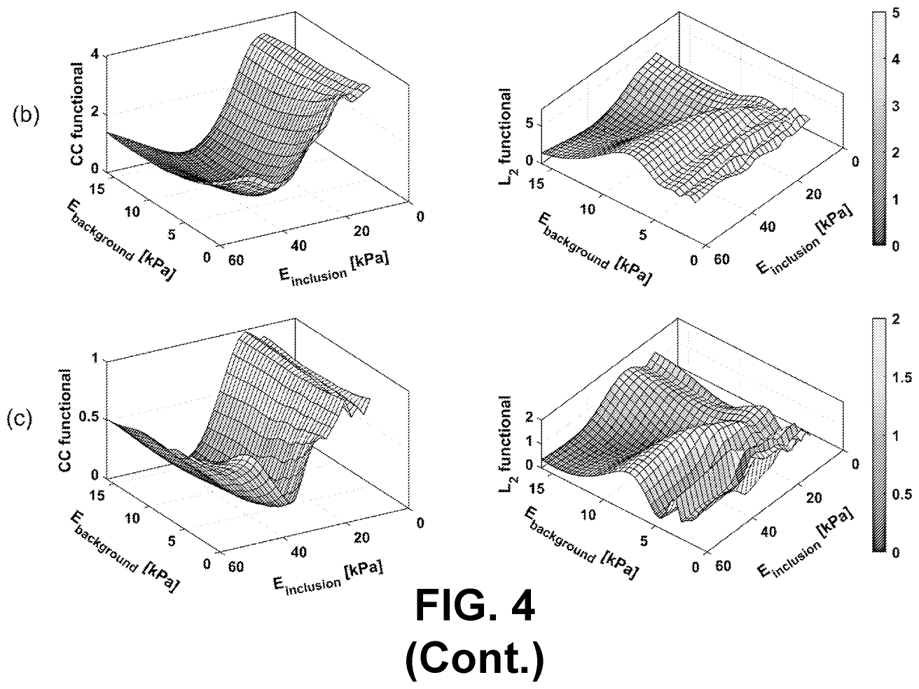

The convergence behavior of L₂ and CC cost functionals are presented in FIG. 3, which illustrates the relative error (left) and gradient (right). In addition, the shapes of the cost functions are shown in FIG. 4, which illustrates a comparison between L₂ functional and the proposed correlation functional: (a) considering noise-free solution and using the entire frequency range, (b) after introducing noise, (c) noise-free solution limited to a single frequency of 500 Hz (this is possible because the current example involves two parameters). Clearly, the terrain of the CC functional is less complex and more convex than that of the L₂. This translates to faster convergence leading to increased efficiency and robustness when CC functional is utilized.

Effect of push amplitude error. To test the reconstructability using the two functionals when the amplitude of the incident wave is not precisely known, an error in the amplitude of the incident wave was intentionally introduced. Plot (b) of FIG. 4 shows the shapes of the two functionals with the erroneous push amplitude. As expected, the CC functional has not been altered, indicating that the global minimum remains at the correct location (background modulus=12 kPa, inclusion modulus=36 kPa). On the other hand, the global minimum for L₂ functional is naturally altered, which corresponds to the incorrect (background modulus=14 kPa, inclusion modulus=35.1 kPa). As expected, the reconstruction using Algorithm 1.1 is successful in estimating the true values of both the background and inclusion when using CC functional, whereas it fails when conventional L₂ functional is used.

Behavior at higher frequencies. As discussed earlier, FWI is a highly nonlinear problem, thus needs a good initial estimate of the model parameter. A bad initial estimate would result in the optimization being trapped in one of the many local minimums. This problem worsens at higher frequencies as seen in plot (c) of FIG. 4, which shows both functionals computed at 500 Hz. Comparing plots (c) and (a) of FIG. 4, the nonlinearity of the L₂ functional computed at 500 Hz seen in plot (c) is significantly higher compared to that for the entire frequency range (i.e., 100-500 Hz) shown in plot (a). This may be attributed to the phenomenon of cycle-skipping. The worsening nonlinearity, which also exists, is significantly less for the CC functional. Thus, unlike the case of L₂ functional, the problem of converging to a local minimum does not appear to be an issue for CC functional.

Effect of initial guess. Examining FIG. 4, it is clear that the basins of the global minima are wider for CC functional than L₂ functional. This indicates that CC functional will have better chance of convergence to the true values and not get trapped at a local minimum, even when the initial guess is not close to the ground truth.

Homogeneous Inclusion with Known Background Modulus. In this section, consider the estimation of the inclusion modulus (equivalently, shear wave speed), assuming that the background shear wave speed is known from other modalities (e.g., localized shear wave elastography that relies on the local homogeneity assumption, which is often valid for the background). In particular, the robustness of the reconstruction process can be examined with respect to the complexity of inclusion shape, noise that is expected in real data, error in push amplitude and signature, as well as errors in the shape of the inclusion.

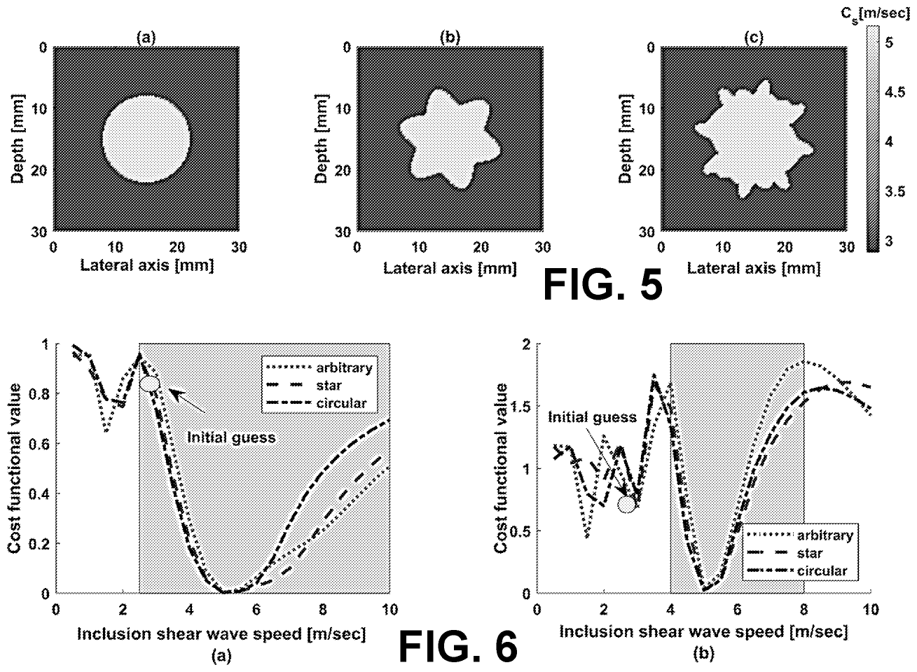

Effect of the complexity of the inclusion shape. Three different inclusion shapes are considered, circular, star and irregular, as shown in FIG. 5. A single frequency of 500 Hz was used for the reconstruction, and 30 synthetically generated measurements on a single line were used for the reconstruction. The initial estimate of the inclusion shear wave velocity was assumed to be the same as that of the background, i.e., 2.88 m/sec.

For all the shapes, CC functional accurately estimates the inclusion shear wave speed, whereas L₂ functional fails in reconstructing the true shear wave speed of the inclusion. This is explained by observing the cost functional plots in FIG. 6, which illustrate the nonlinearity of the cost functional for the three different inclusion shapes when using CC functional (a) and L₂ functional (b). The shaded area indicates the approximate region of expected convergence. Given the initial guess (indicated by the circle), the algorithm with the CC functional (plot (a) of FIG. 6) will converge to the correct solution, whereas it will get trapped at the nearest local minimum when the L₂-functional (plot (b) of FIG. 6) is used. Also, by examining FIG. 6, the CC functional behaves better than the L₂ functional even for the star shaped inclusion.

Effect of noise. Noisy measurement can affect the quality of the reconstructed images. Here, the proposed approach was tested with noisy measurements (synthetic data). To mimic the measurement noise in real data, the synthetic data was polluted with additive Gaussian white noise, with three different signal-to-noise ratios (SNR) of 30, 20, and 10 decibels (dB). The example of irregular inclusion shown in plot (c) of FIG. 5 was utilized, with the same input parameters. Initial estimate of the velocity was taken as the same as the background wave velocity, i.e., 2.88 m/sec. The shape was fixed and inverted for the shear wave speed in the inclusion. Three realizations per each SNR level were inverted and the average reconstructed values are summarized in Table 1.1. For all the SNR levels, the proposed framework was able to reconstruct the shear wave speed in the inclusion with errors less than 0.5%.

TABLE 1.1
Summary of the reconstruction results for different level of noise.

| | True | Reconstructed | |
|---|---|---|---|
| SNR | [m/sec] | [m/sec] | Percentage error |
| 30 dB | 5.16 | 5.14 | −0.4 |
| 20 dB | 5.16 | 5.184 | +0.5 |
| 10 dB | 5.16 | 5.186 | +0.5 |

Effect of error in the assumed inclusion shape. In some situations, the shape of the tumor might not be precisely known. In this case, an approximate shape from a B-mode image may be utilized to start the inversion. Motivated by such scenario, the irregular inclusion in plot (c) of FIG. 5 can be utilized to synthetically construct the measured wavefield (with 10 dB noise), then a circle with a diameter of 14 mm can be used to approximate the true shape and perform the reconstruction. The true shear wave speed of the inclusion is the same as in the previous experiments (i.e., 5.16 m/sec). The reconstructed shear wave speed was 5.49 m/sec which corresponds to a relative error of 6.5%, indicating that the proposed framework is not overly sensitive to error in inclusion shape.

Effect of errors in push configuration, amplitude and location. Often, ARF amplitude and configuration are not precisely known. In addition, there may be an error in the estimated push location. In this section, the effect of errors in push characteristics is explored on the reconstructed results. Consider the irregular inclusion shape shown in plot (c) of FIG. 5 and keep all the inputs the same as in previous examples.

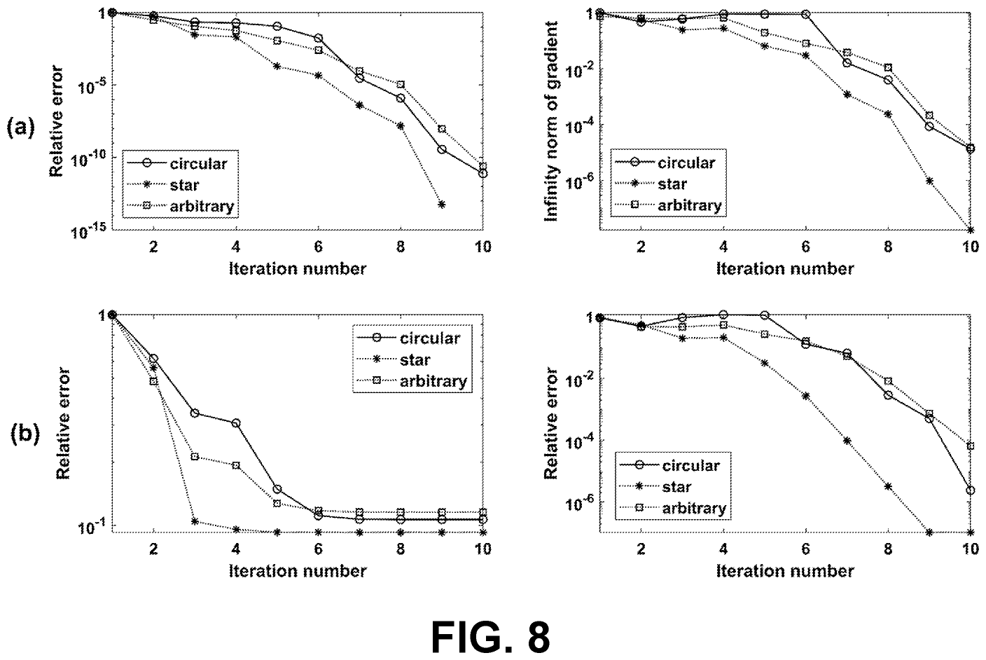

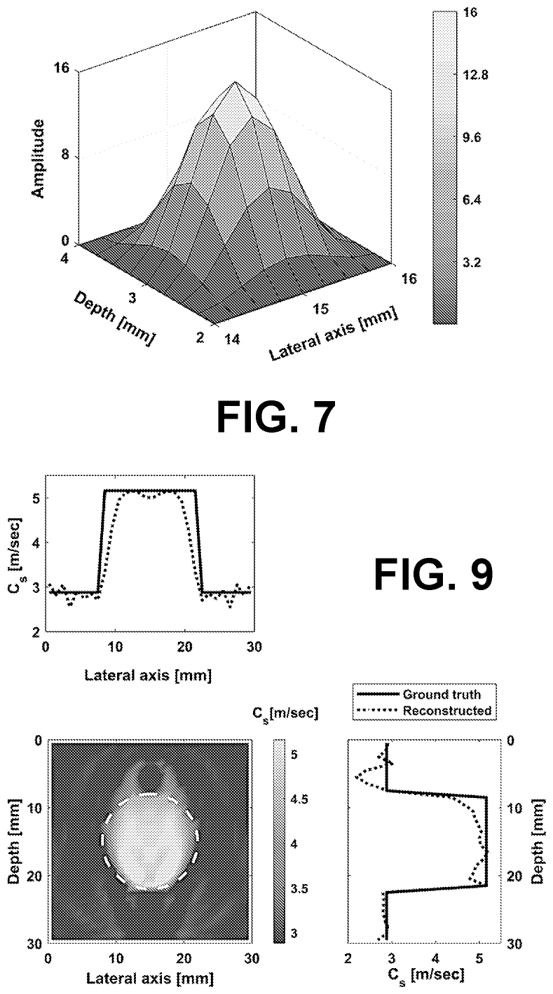

Push signature and amplitude. To mimic errors in push characteristics, consider the modified ARF configuration shown in FIG. 7, which is used for reconstruction (note that this is different in shape and amplitude compared to the default configuration in plot (b) of FIG. 1). Note that in this example, it is assumed that the shape is known, and only the shear wave speed is to be reconstructed. This resulted in the correct reconstructed speed of 5.16 m/sec, which again indicates that the proposed approach is not very sensitive to push signature and amplitude.

Push location. Inject 25% error in the lateral and vertical position of the ARF push. The reconstructed inclusion shear wave speed is 4.76 m/sec, with a relative error of −7.7%, indicating that the reconstruction is not overly sensitive to the push location.

Effect of combined errors. The errors can be combined in the push configuration and location and a 10 dB noise added to the synthetic measurements. In addition, a circle can be used to approximate the irregular inclusion. Reconstruction can be conducted with three separate realizations. The reconstructed inclusion shear wave speeds were 4.95, 5.3, 5.11 m/sec, representing relative errors of −4, 2.7, −0.97%, indicating that combining errors may have cancellation effect. However, error combinations may also have additive effects and it would be best to minimize the input errors to the extent practical.

Homogeneous Inclusion with Unknown Background Modulus. Now consider the case of unknown background shear wave velocity and attempt to simultaneously estimate both the background and inclusion shear wave velocities. The three previous tests were repeated with the same inputs and setup except for three changes: (a) the initial velocity was assumed to be 2.0 m/sec for both background and inclusion, (b) two parameters were inverted, namely the shear wave speeds of the inclusion and the background, (c) the default frequency range (i.e., 100-500 HZ) was used with an increment of 100 HZ. The proposed approach was successful in reconstructing the true shear wave speed of the background and inclusion for all shapes. FIG. 8 illustrate the convergence behavior of two-parameter inversion (a) without noise and (b) with 10 dB noise for the three tests. To test the effect of noise, the reconstruction was repeated after adding the 10 dB noise to the synthetic measurements. The results are summarized in Table 1.2, and the convergence plot is shown in plot (b) of FIG. 8. The relative errors in reconstructed velocities are less than 2%, leading us to conclude that the proposed approach is capable of reconstructing both inclusion and background wave speeds accurately.

TABLE 1.2
Reconstructed shear wave speeds with 10 dB noise.

| | Ground truth | | Reconstructed | | | |
|---|---|---|---|---|---|---|
| | Background | Inclusion | Background | Inclusion | Percentage error | |
| Shape | [m/sec] | [m/sec] | [m/sec] | [m/sec] | Background | Inclusion |
| Circle | 2.88 | 5.16 | 2.84 | 5.12 | −1.4 | −0.78 |
| Star | 2.88 | 5.16 | 2.89 | 5.25 | +0.35 | +1.94 |
| Arbitrary | 2.88 | 5.16 | 2.84 | 5.17 | −1.4 | +0.19 |

Heterogeneous Inclusion. Malignant tumors are often highly heterogeneous. To facilitate inversion of such heterogeneities, the ROI can be split into small pixels, where each pixel has a unique shear wave speed, representing an unknown parameter in the inversion process. In what follows, two examples are presented where the inversion is performed to reconstruct an array of parameters representing the shear wave speed distribution in the entire ROI, or a part of it.

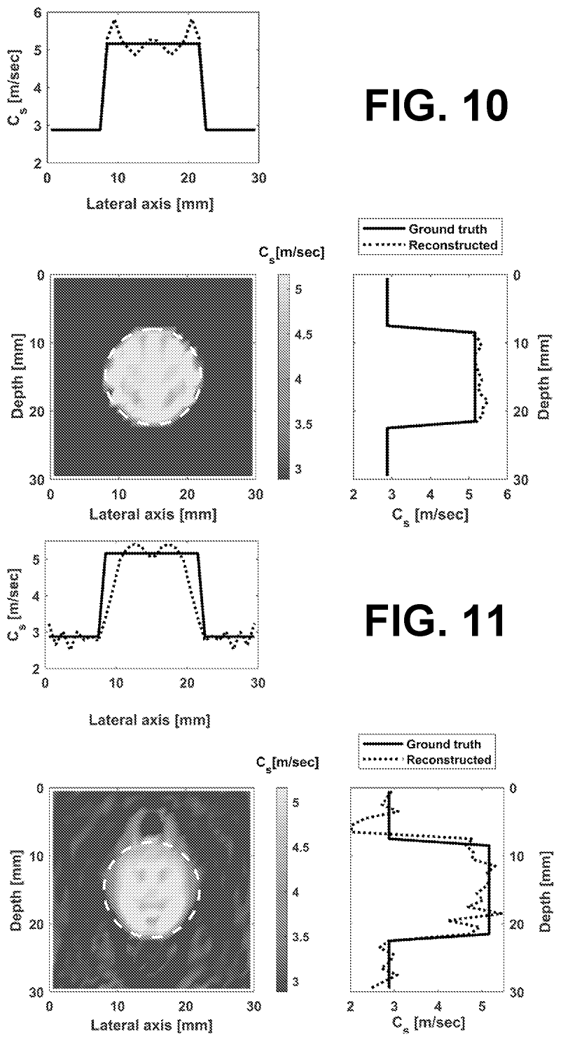

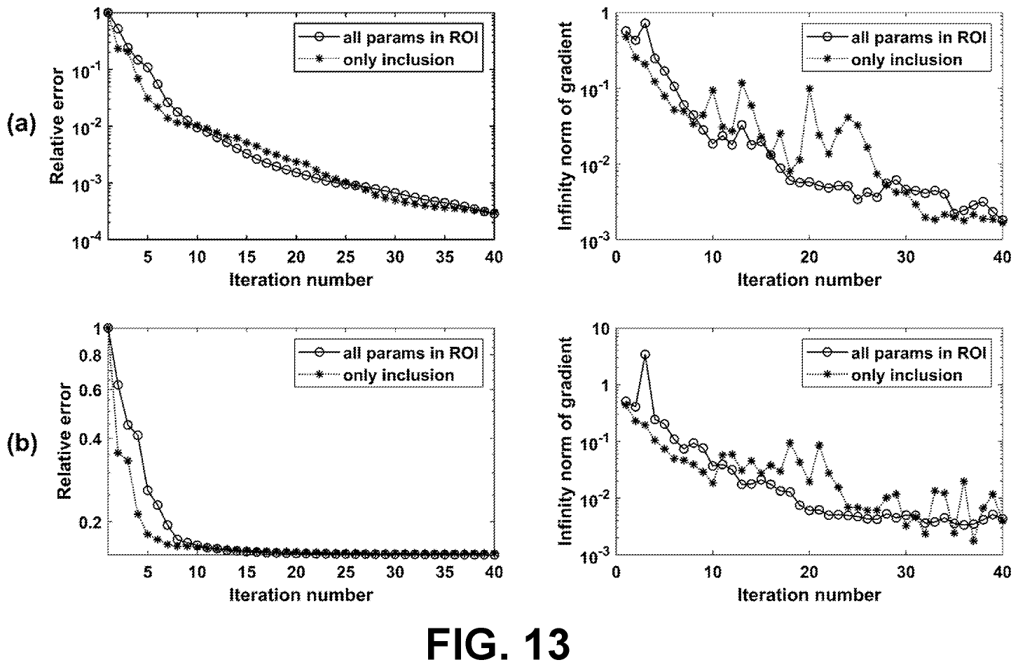

Reconstruction without noise. The performance of the proposed approach was tested for two cases: (i) reconstruct all parameters in the ROI (900 parameters); and (ii) limiting the parameters to be inverted inside a circle with a diameter of 14 mm (same as the inclusion diameter). The frequency range of 100-600 Hz with an increment of 10 Hz was used for inversion (more frequencies were used given the inversion for a much larger number of parameters, otherwise the inversion would be ill-posed). The measurements were considered on one single line with a total of 43 measurements. The initial values of the background and the inclusion were assumed to be 2.88 and 3.6 m/sec, respectively. The reconstructed images are shown in FIGS. 9 and 10, and the convergence behavior for (i) inverting for the entire ROI and (ii) inverting for just the inclusion region without noise are shown in plots (a) of FIG. 13. Left plots contain the cost functional and the right plots contain the norms of the gradient. FIG. 9 illustrates the reconstructed image for case (i): shear wave velocity distribution in the entire ROI. Bottom left is the contour of the shear wave velocity, while the top and side figures contain profiles along the horizontal and vertical diameters. FIG. 10 illustrates the reconstructed image for case (ii): shear wave velocity distribution inside the inclusion (with fixed background shear wave velocity). Bottom left is the contour of the shear wave velocity, while the top and side figures contain profiles along the horizontal and vertical diameters.

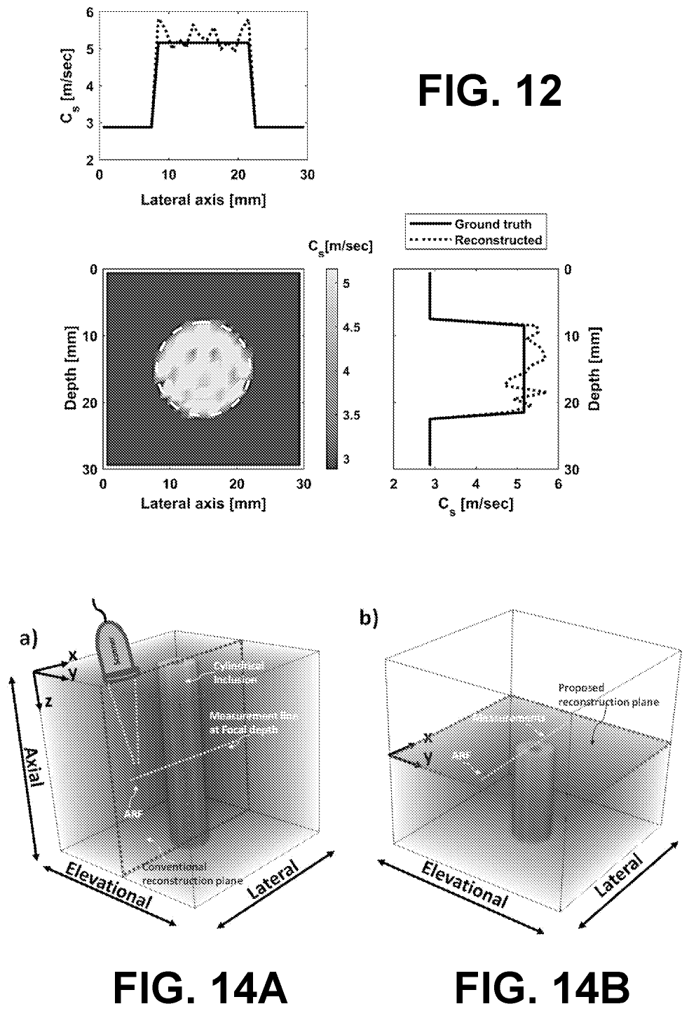

Reconstruction with noise. The reconstruction was repeated after adding 10 dB noise. The reconstruction results for cases (i) and (ii) are shown in FIGS. 11 and 12. FIG. 11 illustrates the reconstructed image for case (i) with noise: shear wave velocity distribution inside the inclusion (with fixed background shear wave velocity). Bottom left is the contour of the shear wave velocity, while the top and side figures contain profiles along the horizontal and vertical diameters. FIG. 12 illustrates the reconstructed image for case (ii) with noise: shear wave velocity distribution inside the inclusion (with fixed background shear wave velocity). Bottom left is the contour of the shear wave velocity, while the top and side figures contain profiles along the horizontal and vertical diameters. The convergence behavior for (i) inverting for the entire ROI and (ii) inverting for just the inclusion region with noise are shown in plots (b) of FIG. 13. Left plots contain the cost functional and the right plots contain the norms of the gradient.

Comparing the reconstructed images with those without noise, more oscillations are seen especially in the vertical sectional profile for noisy data in both cases. The oscillations are more in the axial direction than in the lateral direction, which may be attributed to the measurements being on the vertical line, as explained using the underlying theory of adjoint-based gradient optimization, as follows. The misfit error is oscillatory due to noise and is concentrated on the vertical line as the measurements are restricted to the line. This misfit enters into Equation (1.17) for the adjoint, which in turn enters in Equation (1.16) for the gradient, and then for the image perturbation in Equation (1.10). This process thus leads to oscillatory errors in the image, which are more along the vertical (measurement) line.

A new reconstruction framework has been presented for ultrasound SWE which is based on three main ideas: first, a full-waveform approach is used to better model the wave physics; second, a cross-correlation (CC) based cost functional is used to improve the convergence performance especially with uncertain characteristics of the forcing function; and third, an inversion is performed using measurements on a lower dimensional plane, i.e., use measurements on 1D line to reconstruct an 2D image. The first idea is aimed at mitigating the errors associated with plane wave and homogeneity assumptions of local reconstruction methods (conventional SWE). The second idea is based on the observation that CC functional is more convex than L₂ functional, leading to better convergence behavior. The motivation for the third idea is that a conventional ultrasound transducer can only measure the wavefield in a plane while tumors to be imaged are 3D by nature. An attempt is made to mimic the setting in the simpler lower-dimensional settings where measurements on 1D line is used to reconstruct 2D images.

The CC functional emphasizes mainly phase information, as opposed to both phase and amplitudes that are highlighted by classical L₂ functionals. The CC functional is independent of the push information and is less sensitive to other errors, resulting in a much better behaving cost functional compared to the L₂ functional. This is illustrated in FIG. 6, where the new framework results in a wider radius of convergence compared to that when using the standard L₂ functional. Moreover, FIG. 6 indicates that the CC functional is more robust with respect to increasing the complexity of the inclusion shape. When compared to the L₂ functional, the CC functional succeeded in reconstructing the shear wave speed in the inclusion, for all considered inclusion shapes and with noisy measurements.

A motivation of inverting shear wave speed only in the inclusion is because it is assumed that the properties of the soft tissue surrounding the tumor can be obtained by another modality. Notwithstanding this, synthetic experiments can be used to reconstruct both background and inclusion shear wave speed, and the methodology can be as effective as an estimation of the shear wave speed in the inclusion. Finally, the proposed inversion can be applied to image heterogeneous tumors, leading to promising results, especially with known background properties.

Note that although all the measurements are synthetically generated, there are cases where there is not a complete match between the reconstructed and true values/images. This may be attributed to solving an ill-posed nonlinear optimization problem with gradient-based algorithm, thus, the reconstruction depends on many aspects such as the initial guess, parameterization, and the amount of data available. Therefore, it is not expected to have a complete match between the reconstructed shear wave speed distribution and the true values, unless the inversion is for just a handful of parameters.

For evaluating cost functional and the gradient, the proposed approach with CC functional needs almost the same computational effort as that using conventional L₂ functional. Additionally, given the improved behavior of the CC functional leading to faster convergence, the proposed approach is often faster than L₂ approach. With respect to the absolute computational cost, inversion for a single elastic modulus of the inclusion took less than a minute on a desktop computer with Intel Xeon W2295 CPU and 128 GB RAM (note that the implementation is preliminary, and no significant optimization was performed). For the most complex case of inverting 900 parameters using 51 frequencies and 4761 degrees of freedom, it took about 10 minutes on the same computer. The run time may be significantly reduced using code optimization and utilizing the parallel nature of the forward model, where each frequency can reside on a separate processor.

This work is based on 2D anti-plane shear approximation of the wave propagation in an elastic medium. In reality, tumors are 3D in nature, where the tissue can be considered incompressible. Thus, to extend the framework to 3D where the objective would be reconstructing the 3D distribution of the shear wave speed. The main complexity here would be efficient forward modeling in 3D, considering incompressible elasticity. The ensuing mathematical formulation for adjoint-based gradient optimization can translate naturally from that presented here.

### Multi-Acquisition Multi-Resolution Full-Waveform Shear Wave Elastography of Soft Tissue Viscoelasticity

Soft tissue's mechanical characteristics are strongly linked to physiology and pathology. For example, a change in the elasticity (stiffness) of the soft tissue may be an indication of an onset of a health problem or a disease, e.g., cancer. Over the last three decades, scientists have invented several modalities to reconstruct maps of soft tissue elasticity. Among them, magnetic resonance imaging (MRI), computed tomography CT, ultrasound imaging UI, or optical coherence tomography OCT, among others. Although each of these modalities show success in particular applications, ultrasound imaging, which is the focus of this work, has an increasing interest because it is cheaper and safe. Several existing algorithms reconstruct only elasticity and not viscosity, however, there is increasing evidence that soft tissue viscosity can add valuable information to disease diagnosis. Further, some diseases may exhibit a strong heterogeneity in stiffness and/or viscosity, e.g., cancerous tumors or exhibit a diffused heterogeneity in the viscoelastic properties, e.g., diffuse liver diseases. Thus, it is of significant interest to develop a methodology that not only could reconstruct accurate elasticity map but also viscosity.

Several efforts have been made to reconstruct a complete viscoelasticity map of soft tissue. Methods based on shear wave elastography SWE based on acoustic radiation force (ARF) are widely used to estimate viscoelasticity maps. Supersonic shear imaging, the local phase velocity imaging, the harmonic motion imaging, the frequency-shift imaging, the local model-based shear wave dynamics and the shear wave dispersion ultrasound vibrometery SDUV and others are among the point estimation and imaging methods based on SWE-ARF. Most of these methods adopt several idealizations of the underlying wave physics, e.g., local homogeneity and plane wave assumption, thus ignoring the complex, yet important, wave propagation characteristics (i.e., reflections, diffractions). This limitation has been recently tackled for elasticity and significant imaging capability provided out of the measurement plane.

In contrast to reconstructing the elasticity, it has been reported that reconstructing viscosity is a challenging task, especially when reconstructing both parameters (elasticity and viscosity) simultaneously. This may be attributed to the fact that there is a strong dependence between the elasticity and viscosity in a phenomenon called parameter crosstalk. In addition, the uncertainty related to the underlying viscoelastic constitutive model poses another difficulty when reconstructing a complete viscoelasticity map of soft tissue.

There has been extensive research conducted to mitigate the crosstalk between the parameters. To reconstruct a good viscosity map, it is important to have a very good velocity map (shear modulus map) which ensures accurate kinematics of the reconstruction (e.g., arrival time). The opposite is not possible (i.e., constructing a viscosity map first then elasticity) because there is evidence that the footprint of the viscosity, which is translated to data as amplitude attenuation, is very small compared to the footprint of the shear modulus, thus hindering accurate reconstruction of viscosity when elasticity map is not accurate.

To this end, a goal of this work is two-fold. First, develop a robust methodology of reconstructing shear modulus distribution map as accurately as possible (by enhancing the methodology by utilizing acquisitions from multiple pushes. Then extend the methodology to reconstruct the viscosity, which is shown to be effective, consistent with the hypothesis that capturing the kinematics of wave propagation would help lead to good convergence for viscosity inversion.

METHODS. In this section, details of the methodology are presented starting with the forward modeling of the wave physics in a viscoelastic soft tissue. Then details of the optimization framework were presented where the objective function and its gradient were derived in detail. Several methodologies are presented that show promise in improving the ability to reconstruct the complete viscoelasticity maps of soft tissues.

Forward model. The soft tissue can be modeled as an isotropic, linearly viscoelastic medium. The assumption of linearity follows the fact that ARF is generally very low in amplitude that makes the material nonlinearity not enter into the picture. In addition, any change in the material properties that may result from the change in the fluid pressure after the application of the ARF may be ignored. The partial differential equation (PDE) that governs wave propagation in an isotropic incompressible viscoelastic medium is given by the electrodynamics equation:

$$- ∇ \cdot σ ( \underline{x} , t ) + ρ \ddot{u} ( \underline{x} , t ) = f ( \underline{x} , t ) δ ( \underline{x} , - {\underline{x}}_{o} ) , \underline{x} ∈ {Ω}_{ROI} ∈ {R}^{3} , \qquad ( 2.1 )$$

where σ is the stress tensor, f represent the acoustic radiation force (ARF), δ is Dirac function, x is the position vector, xₒ is the position of the ARF which is idealized as a concentrated push, ρ is the tissue density, typically taken as that of water ρ=1000 kg/m³ and a is the particle acceleration.

In this study, the Kelvin-Voigt model is discussed as the underlying viscoelastic constitutive model to describe the viscoelastic behavior of the soft tissue, but the method is general and other viscoelastic constitutive relation can be directly applied. The stress-strain relationship can be given by the following equation:

$$σ = {σ}_{e} + {σ}_{v} = B ( G ) ε + C ( η ) \dot{ε} \qquad ( 2.2 )$$

where (σₑ, σᵥ) are the elastic and viscous stresses, respectively, B is the elastic modulus tensor, C is the =viscosity tensor, which can be written in terms of shear modulus G and shear viscosity η, respectively. \dot{ε}=∂E/∂t is the strain rate.

FIG. 14A schematically illustrates an example of the ultrasound imaging setup of the acquisition setup in a 3D view and FIG. 14B illustrates the proposed reconstruction in the x-y plane. The dotted plane in FIG. 14A represents the conventional imaging and reconstruction plane, whereas the plane in FIG. 14B represents the proposed reconstruction plane. The dotted-white line represents the measurements considered in this study.

Note that, while the imaging domain is three-dimensional (3D), e.g., FIG. 14A, in this work, the feasibility of the methodology was investigated in a two-dimensional (2D) version with a simpler setup that still captured the main complexity, e.g., wave scattering out of the measurement plane. Specifically, the imaging domain was projected on a 2D horizontal plane (x-y plane) with the goal to reconstruct 2D maps from anti-plane shear movements on a line (1D plane), see FIG. 14B. This is in contrast with many of the existing methods that consider the imaging plane in the x-z plane, see FIG. 14A.

In ARF-based shear wave elastography, the push direction is always in the (z) direction (vertical), i.e., out-of-plane w.r.t FIG. 14B. Therefore, Equation (2.1) can be reduced to be the anti-plane shear wave equation in a 2D viscoelastic medium. Therefore, only the shear modulus G (kPa) and shear viscosity η (Pa·s) are needed to fully describe the underlying viscoelastic constitutive relationship. Note that the proposed methodology is general and could be extended to 3D, however, in this study we will limit the discussion to 2D modeling. As mentioned earlier, most of the soft tissues are nearly incompressible (i.e., v→0.5), which means G=E/3. Note that there is no volumetric locking issue even if a linear finite element were to be used for the forward modeling because we are solving an anti-plane shear wave equation.

In this study, Equation (2.1) was solved numerically in frequency domain so that better control was achieved on the numerical dispersion resulting from the numerical discretization especially at higher frequencies. In addition, the solution process would be computationally more efficient if a proper set of frequencies were chosen. Finally, the inversion approach utilizes frequency-based multiresolution approach, necessitating frequency-domain forward modeling. The frequency domain PDE is obtained by Fourier transforming Equation (2.1) in time:

$$∇ \cdot \overline{σ} ( \underline{x} , ω ) + f ( \underline{x} , ω ) δ ( \underline{x} - {\underline{x}}_{o} ) = - {ρω}^{2} \overline{u} ( \underline{x} , ω ) , \underline{x} ∈ {Ω}_{ROI} , \qquad ( 2.3 )$$

where ω is the angular frequency and (σ̅, f̅, ū) are the stress tensor, the ARF and the anti-plane particle displacements in frequency domain. Further assume that the tumor is far from the surface and other physical boundaries. Given these assumptions, consider the background of the ROI to be homogenous and waves travel outside the ROI without being reflected back to the ROI. Therefore, a radiation boundary condition was utilized on the boundary of the ROI (∂Ω). The boundary condition can be formally written as,

$$\overline{σ} \cdot n = Λ \overline{u} \qquad ( 2.4 )$$

where n is the unit normal to the boundary surface ∂Ω and Λ is the Dirichlet-to-Neumann operator representing an absorbing boundary condition. The absorbing boundary condition in Equation (2.4) can be modeled in several forms, starting from the simplest, where only the normal and tangential incidents are absorbed, to the most complex case where incidents in a wide-angle range are absorbed. While efficient versions Λ would make the absorption more accurate (e.g., PML, PMDL), the simpler Sommerfeld radiation boundary condition was utilized.

$${lim}_{\vert {x}_{j} \vert \to ∞} ( \frac{\partial }{\partial {x}_{j}} - i {k}_{j} ) \overline{u} ( {x}_{j} ) = 0 , \qquad ( 2.5 )$$

where kⱼ is the wavenumber, xⱼ is spatial direction, with j=1, 2 for two-dimensional problems.

Since ARF amplitude is very low, soft tissue undergoes only small deformation due to the ARF application. Therefore, consider the small deformation theory where,

$$ε = \frac{1}{2} ( ∇ \overline{u} + ∇ {\overline{u}}^{T} ) . \qquad ( 2.6 )$$

Therefore, the strong form of the boundary value problem is:

$$∇ \cdot \overline{σ} + \overline{f} δ ( \underline{x} - {\underline{x}}_{o} ) = - ρ {ω}^{2} \overline{u} , \underline{x} ∈ {Ω}_{ROI} , \qquad ( 2.7 )$$

$$\overline{σ} = ( \overline{B} ( G ) + i ω \overline{C} ( η ) ) ε = \overline{D} ( G , η ) ε and ε = \frac{1}{2} ( ∇ \overline{u} + ∇ {\overline{u}}^{T} ) ,$$

$$\overline{σ} \cdot n = Λ \overline{u} on \partial Ω$$

where D̅(G, η) is the viscoelasticity tensor and ( ) represents the frequency domain version of the function/tensor.

After re-arranging Equation (2.7) and multiplying it by a virtual function υ, the weak form of Equation (2.7) can be written as:

$$( \overline{u} ) = {\int }_{{Ω}_{ROI}} \overline{σ} ( \overline{u} ) : ε ( υ ) - {\int }_{\partial Ω} ( \overline{σ} ( \overline{u} ) \cdot n ) υ + {ρω}^{2} {\int }_{{Ω}_{ROI}} \overline{u} υ + {\int }_{{Ω}_{ROI}} \overline{f} δ ( \underline{x} - {\underline{x}}_{o} ) υ = 0 , \qquad ( 2.8 )$$

$$∀ υ ∈ V := \{ υ : υ ∈ {H}_{0}^{1} ( {Ω}_{ROI} ) \} .$$

In this work, the finite element method (FEM) was used to Equation (2.8). Specifically, the Multi-physics Object Oriented Simulation Environment (MOOSE) was used, where the first term in the residual is modeled using the stress divergence kernel, the second term is the traction boundary conditions, the third term is modeled using reaction kernel and the last term is modeled using the Dirac kernel. Further, the 2D bilinear finite element was adopted with linear shape function for modeling the entire ROI.

Reconstruction through PDE-constrained optimization. Reconstruction aims to infer the distribution of shear modulus and viscosity, represented by parameter vector p, by minimizing an objective functional, [symbol] (p) that is a measure of the mismatch between the measurements and predicted (simulated) responses. In this work, a PDE-constrained optimization framework was adopted and an objective function devised, which is iteratively minimized using adjoint based gradient optimization. The PDE-constrained optimization problem can then be written as:

$$\hat{p} = \begin{matrix}arg min \\ p\end{matrix} ( p ) \qquad ( 2.9 )$$

$$Subjected to ( \overline{u} , p ) = 0$$

where \hat{p} is the optimal model parameter that minimizes the objective function [symbol] (p), while [symbol] is the governing PDE constraint, defined in Equation (2.7). In this work, a cross-correlation (CC) functional developed in previous elasticity imaging work will be utilized:

$$𝒥 ( p ) = 1 - \frac{{\vert d \cdot ( 𝒫 \overline{u} ( p ) ) \vert}^{2}}{{( ‖ d ‖ ‖ 𝒫 \overline{u} ( p ) ‖ )}^{2}} \qquad ( 2.1 )$$

where [symbol] is the measurement vector obtained on a subset of the ROI, i.e., Ω_{data}⊆Ω_{ROI}, a is the simulated wavefield, and [symbol] is a projection operator that restricts a to the measurement set.

Newton-type algorithms minimize second order Tylor expansion at each iteration, i.e., the objective function is approximating as,

$$( {p}_{0} + Δp ) \approx ( {p}_{0} ) + Δ {p}^{T} g + \frac{1}{2} Δ {p}^{T} H Δ p \qquad ( 2.11 )$$

where g is the gradient vector, H is the Hessian matrix and T represents matrix transposition. Assuming that the Hessian is invertible, the estimate of the perturbation Δp which minimizes the functional in Equation (2.11) can be determined by,

$$Δ p = - {H}^{- 1} g . \qquad ( 2.12 )$$

for a handful number of model parameters, the finite difference approximation can be utilized for the gradient computation (by perturbing each parameter). However, when the number of model parameters is large, taking finite differences by perturbing each parameter will be highly inefficient. Instead, the adjoint formulation can be utilized for the gradient calculation.

Gradient computation through adjoint formulation. The gradient g of the objective functional [symbol] (p) is the total derivative of [symbol] with respect to its argument p, which can be written using chain rule as, this can be written as,

$$g ( p ) = \frac{d ( p )}{( i p} = \frac{\partial ( p )}{\partial p} + \frac{\partial ( p )}{\partial \overline{u}} \frac{\partial \overline{u}}{\partial p} . \qquad ( 2.13 )$$

Noting that [symbol] (ū, p)=0, and thus d [symbol] (ū, p)/dp=0 and using chain rule,

$$\frac{d ( \overline{u} , p )}{dp} = \frac{\partial ( \overline{u} , p )}{\partial p} + \frac{\partial ( \overline{u} , p )}{\partial \overline{u}} \frac{\partial \overline{u}}{\partial p} = 0 , \qquad ( 2.14 )$$

leading to,

$$\frac{\partial \overline{u}}{\partial p} = - {( \frac{\partial ( \overline{u} , p )}{\partial \overline{u}} )}^{- 1} \frac{\partial ( \overline{u} , p )}{\partial p} , \qquad ( 2.15 )$$

Substituting the above in Equation (2.13) results in the gradient expression:

$$\frac{d ( p )}{dp} = - \frac{\partial ( p )}{\partial \overline{u}} {( \frac{\partial ( \overline{u} , p )}{\partial \overline{u}} )}^{- 1} \frac{\partial ( \overline{u} , p )}{\partial p} , \qquad ( 2.16 )$$

Introduce the adjoint variable λ as the solution of the adjoint equation,

$${( \frac{\partial ( \overline{u} , p )}{\partial \overline{u}} )}^{T} λ = {[ \frac{\partial ( p )}{\partial \overline{u}} ]}^{T} , \qquad ( 2.17 )$$

the gradient expression in Equation (2.13) takes the final form,

$$g ( p ) = - {λ}^{T} \frac{\partial ( \overline{u} , p )}{\partial p} . \qquad ( 2.18 )$$

Practically, in variational settings of the forward problem in Equation (2.8), the gradient is computed as the weighted inner product of forward and adjoint strains, ε(ū) and ε(λ), as seen from the following equation:

$$g ( p ) = - {λ}^{T} \frac{\partial ( \overline{u} , p )}{\partial p} = {\int }_{{Ω}_{ROI}} {ε ( λ )}^{T} \frac{\partial \overline{D} ( G , η )}{\partial p} ε ( \overline{u} ) . \qquad ( 2.19 )$$

The weighting tensor is essentially with the derivative of the viscoelasticity tensor with respect to the model parameter p.

It is worth noting that the kernel

$$\frac{\partial ( \overline{u} , p )}{\partial p}$$

independent of the choice of the objective functional [symbol] and the measurement data, on the other hand, the adjoint variable λ is dependent on the choice of [symbol] . Adjoint formulation is widely used for solving inverse problems, and we have adopted such formulation in our recent work on elasticity imaging.

Parametrization. The inversion parameters (shear modulus and viscosity) must be positive quantities. Thus, when not applying any bound constraints on the parameter, there is a high chance of getting non-physical values (e.g., −ve). Alternatively, in this work, the parameters can be transformed to the log-scale and invert for p̅:={log G, log η}. Note that, for Kelvin-Voigt viscoelastic constitutive model, the complex shear modulus D̅(G, η) is given by,

$$\overline{D} ( G , η ) = G + i ωη \qquad ( 2.2 )$$

leading to the following expressions for the derivatives D̅ with respect to the two parameters:

$$\frac{\partial \overline{D} ( G , η )}{\partial ( log G )} = ln ( 10 ) {log}_{10} ( G ) , \frac{\partial \overline{D} ( G , η )}{\partial ( log η )} = i ωln ( 10 ) {log}_{10} ( η ) . \qquad ( 2.21 )$$

Comment on parametrization: While Kelvin-Voigt model in Equation 2.20 was utilized for illustrating the approach, various types of viscoelastic behavior can be handled with the developed approach. These include Burger's, spring-pot, fractional Voigt, as well as general viscoelastic models, e.g. those based on Prony series that can fit frequency-dependent complex modulus.

Optimization algorithm. Casting the problem as PDE-constrained optimization and use gradient-based optimization framework for the reconstruction. There are several gradient-based optimization algorithms that can be used. Based on experience with seismic and shear wave elastography imaging problems, BFGS algorithm can be utilized, which turned out to perform well for the numerical examples explored in this work. The steps are presented in Algorithm 2.1.

Algorithm 2.1: Proposed reconstruction algorithm based on CC cost functional.

```
- Given the measurements d.
- Discretize the ROI into small pixels representing shear modulus and shear viscosity
  spatial distribution p:={G, η}.
- Given an initial model p₀ and stopping criterion e.g., ∥g∥ ≤ ε, minimize the cost
  functional in Equation (2.10) over all frequencies and pushes until convergence. Each
  iteration k of the minimization process involves the following steps:
  5. Obtain the preliminary step Δpₖ in Equation (2.12) using the following steps:
    d. Compute the adjoint variable 2 in Equation (2.17). This requires one adjoint
      solve.
    e. Compute the gradient gₖ using Equation (2.19).
    f. Solve Equation 2.12) using the BFGS algorithm to get the step Δpₖ.
  6. Using cubic line search, minimize the cost function in Equation (2.11) to compute
    the step size α.
  7. Update the model:
    b. \hat{p}=pₖ + αₖΔpₖ.
  8. Repeat step 1 until ∥gₖ∥ < ε.
```

Multiresolution imaging through expanding frequency windows. Full waveform inversion described in Equation (2.11) is a nonlinear optimization problem which usually exhibits highly nonlinear behavior with respect to the parameters. In addition, the problem is ill-conditioned due to the scarcity of the data. This results in optimization being easily trapped in one of the many local minima. The problem is even worse at high frequency regime, making it harder to obtain accuracy in high resolution imaging. To address this issue, a good initial estimate would be needed. However, obtaining a good initial estimate is not usually an easy task because it depends on the operator's expertise and prior knowledge, and/or the accuracy of other ultrasound modalities used for initial imaging (e.g., B-mode image). Another way is to regularize the objective function with a proper regularization term. Finding the proper regularization term is not trivial and a problem-specific, especially for FWI problem that involves multi-parameter inversion (e.g., elasticity and viscosity). The choice of the regularization scheme and its optimal parameter are even more challenging in in-vivo applications. Motivated by this, take advantage of performing the reconstruction in the frequency domain and adopt the multi-resolution/stage strategy for imaging the elasticity and viscosity maps simultaneously. Specifically, first, reconstruct lower resolution images utilizing the lower frequency content in the measurements, then, use the spatial mean (average) of the lower resolution reconstructed images as an initial guess for the next stage (higher resolution) (i.e., by including higher frequency content). It is demonstrated through several examples that this strategy is promising in reconstructing both shear elasticity and viscosity in a tumor-like tissue.

Algorithm 2.2: Multi-resolution reconstruction algorithm based on CC cost functional.

```
- Obtain measurements on a line d.
- Discretize the ROI into small pixels representing shear modulus and shear viscosity
  spatial distribution p:={G, η}.
- Divide the reconstruction to n group of frequencies (resolutions), set a stopping
  criterion e.g., ∥g∥ ≤ ε, and perform the following:
- Use the results of the preceding resolution as a starting model p₀, minimize the
  cost functional in Equation (2.10) over the frequencies in this group. Each iteration k
  of the minimization process involves the following steps:
  1. Obtain the preliminary step Δpₖ in Equation (2.12) using the following steps:
    g. Compute the adjoint variable λ in Equation (2.17). This needs one adjoint
      solve.
    h. Compute the gradient gₖ using Equation (2.19).
    i. Solve Equation (2.12) using the BFGS algorithm to get the step Δpₖ.
  2. Using cubic line search, minimize the cost function in Equation (2.11) to compute
    the step size α.
  3. Update the model:
    c. \hat{p}=pₖ + αₖΔpₖ.
  4. Repeat step 1 until ∥gₖ∥ < ε.
```

Joint reconstruction of elasticity and viscosity. Due to the crosstalk between the shear elasticity and viscosity, it is intrinsically difficult to jointly reconstruct both parameters (shear elasticity and viscosity). In addition, there is evidence that the footprint of the viscosity, which is translated as amplitude attenuation in the data, is very small compared to the footprint of the shear modulus, thus hindering accurate reconstruction of viscosity when elasticity is not accurately reconstructed. Consequently, to reconstruct a good viscosity map, it is important to have an excellent velocity map (shear modulus map) which ensures accurate kinematics of the reconstruction (e.g., arrival time). To this end, Algorithm 2.2 can be updated to initially allow for reconstructing both parameters, where allowing for viscosity inversion would add more degree-of-freedoms (DOFs) to the optimization problem (better inversion for shear elasticity). However, the expectation is that the reconstructed viscosity would not be reliable (for the same reasons discussed earlier). Once a good shear modulus map is obtained, fix that map, and only invert for viscosity with the expectation to get a better viscosity map.

Multi-acquisition. In several circumstances, the target region to be reconstructed is relatively large for a single acquisition to reliably reconstruct its mechanical properties. To mitigate this problem, inversion can be done by combining multiple acquisitions resulting from multiple pushes. This is because having more data/information at different positions/location would make it possible to illuminate the whole target region. Especially for heterogeneous targets, this could give a better chance of reconstructing accurate images as we demonstrate in the Results section.

Restricting inversion to a spatial window. Reconstructing the whole region of interest (ROI) would be of interest in some cases, however, in majority of the cases, the reconstruction is only needed for a particular target (part of the ROI), e.g., tumor. The expectation is that the mechanical properties of background are usually known or can be easily determined using point SWE algorithms. In this work, it is possible to take advantage of this and only restrict the reconstruction to a window that is smaller than the ROI, leading to a reduction in the number of parameters to be optimized (less unknowns). This, in fact, would make the optimization process less ill-posed, and make it more possible to use fewer number of acquisitions. The size of the reconstruction window can be then determined by examining the B-mode images. However, note that there is uncertainty in B-mode images, e.g., tumor size. For example, B-mode images often depict smaller tumor size. Therefore, in what follows, the reconstruction can be limited to a window (circular window) of a diameter that is larger than the expected inclusion diameter.

RESULTS. Consider reconstructing the shear modulus and/or viscosity using the algorithms presented in the previous section, and the setup in FIG. 14A. An attempt to reconstruct an out-of-plane image with respect to the acquisition plane is shown in FIG. 14B. Thus, all the images presented in this section are in the x-y plane. In all of the examples, the aim was to reconstruct the complete viscoelasticity map of a circular inclusion with diameter of 8 mm, with SWE data ranging from 50-650 Hz with increment of 100 Hz. The synthetic measurements were sampled every 0.5 mm on the mid-line shown in FIG. 14B. The measurements on a line at x=15 mm were considered, and only points from y=0.5 mm to 19.5 mm were considered in order to reduce the effect of the push on the inversion (see FIG. 14A). The background shear modulus and viscosity were used as initial guess for inclusion shear modulus and viscosity. The optimization was stopped when the infinity norm of the gradient reached 0.01% of the initial gradient. In this section, start by jointly reconstructing both shear modulus and shear viscosity maps where it was concluded that it is difficult to reliably reconstruct a heterogeneous viscoelasticity map. Then, in the next section, a multiresolution strategy where first an accurate shear modulus map was reconstructed, is fixed and used for shear viscosity reconstruction.

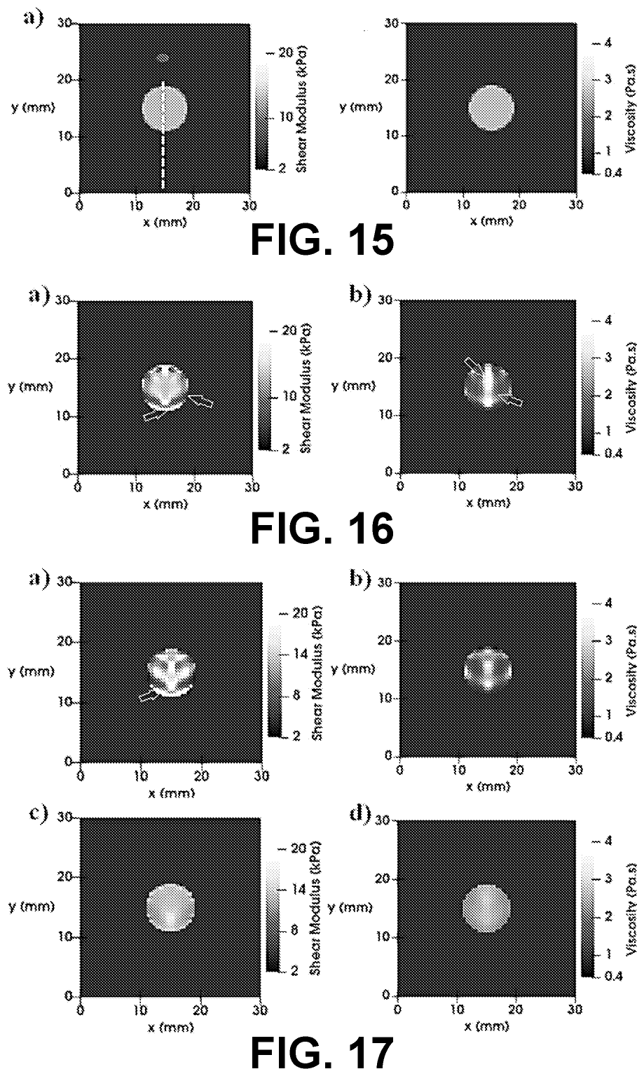

Reconstruction with single push. In this section, consider the reconstruction of the shear elasticity and viscosity maps given measurements on a single line from a single push (single acquisition). This example evaluates the proposed algorithm when only one acquisition is available. The true shear elasticity and viscosity maps are shown in plots (a) and (b) of FIG. 15, respectively. The dotted-white line indicates the measurements line, while the red ellipse represents the ARF. The reconstructed maps of the shear elasticity and viscosity (reconstruction with single acquisition: shear modulus distribution and shear viscosity distribution) are shown in plots (a) and (b) of FIG. 16, respectively. The white arrows indicate the location of some artifacts.

Cleary, it can be observed that the reconstructed shear elasticity is close to the true shear elasticity indicating the promise of the method in reconstructing the shear elasticity maps even in the existence of viscosity. However, the viscosity map is not as good as the shear elasticity map. This may be attributed to several issues, including the parameter cross-talks mentioned earlier, and the limited availability of measurements, all of which increase the ill-posedness of the inverse problem. Thus, the results when using the multi-resolution reconstruction are presented.

Reconstruction with multi-resolution approach. Although the resulting maps in FIG. 16 are promising, especially the shear modulus map, several artifacts can be seen in both maps, especially in the viscosity map. These artifacts are an indication that optimization is getting trapped in several local minima. This issue can be addressed by using the described multi-resolution approach and outlined by Algorithm 2.2. Applying this strategy to the previous example, and significant improvement could be seen not only in the shear modulus map, but also in the shear viscosity map. FIG. 17 illustrates the reconstructed shear modulus and viscosity maps through multi-resolution reconstruction. Plots (a) and (b) show reconstructed maps for 50-350 Hz and plots (c) and d) show reconstructed maps for 50-650 Hz. The white arrows indicate the location of some artifacts.

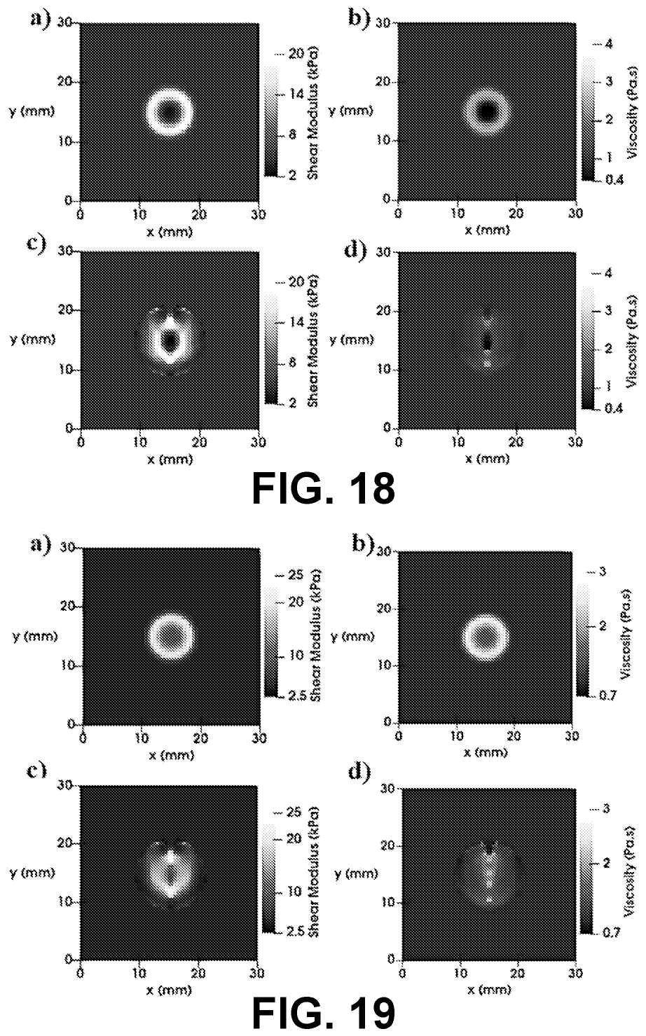

The question that would arise is whether this strategy can be applied to more challenging cases, where for example, the disease is heterogeneous, i.e., the shear modulus and viscosity are locally varying rapidly. In the following examples this concern is addressed, and the multi-resolution strategy applied with spatial averaging to a case of a heterogeneous tumor. In these examples, assume that the inside of the tumor is softer than the periphery. The viscosity of the tumor is also heterogeneous as shown in FIG. 18, which illustrates a reconstruction of a tumor-like tissue (tumor I). Plots (a) and (b) show the true shear modulus and viscosity maps and plots (c) and (d) show the reconstructed maps. The reconstruction window was limited to a circle of a diameter of 12 mm, larger than the inclusion but smaller than the entire ROI. FIG. 19 illustrates a reconstruction of a tumor-like tissue (tumor II). Plots (a) and (b) show the true shear modulus and viscosity maps and plots (c) and (d) show the reconstructed maps.

Using two examples of tumor heterogeneities (tumor I, see e.g., plots (a) and (b) of FIG. 18, and tumor II, see e.g., plots (a) and (b) of FIG. 19), it is demonstrated that it is possible to recover the overall distribution of the shear modulus, whereas we see difficulties in reconstructing viscosity as seen in plots (c) and (d) of FIG. 18 and plots (c) and (d) of FIG. 19. Motivated by this, consider the more challenging case of heterogeneity shown in plots (a) and (b) of FIG. 19 (i.e., tumor II) and in-line with the multi-resolution methodology presented here, and using multiple acquisitions to improve the reconstructed viscoelasticity maps.

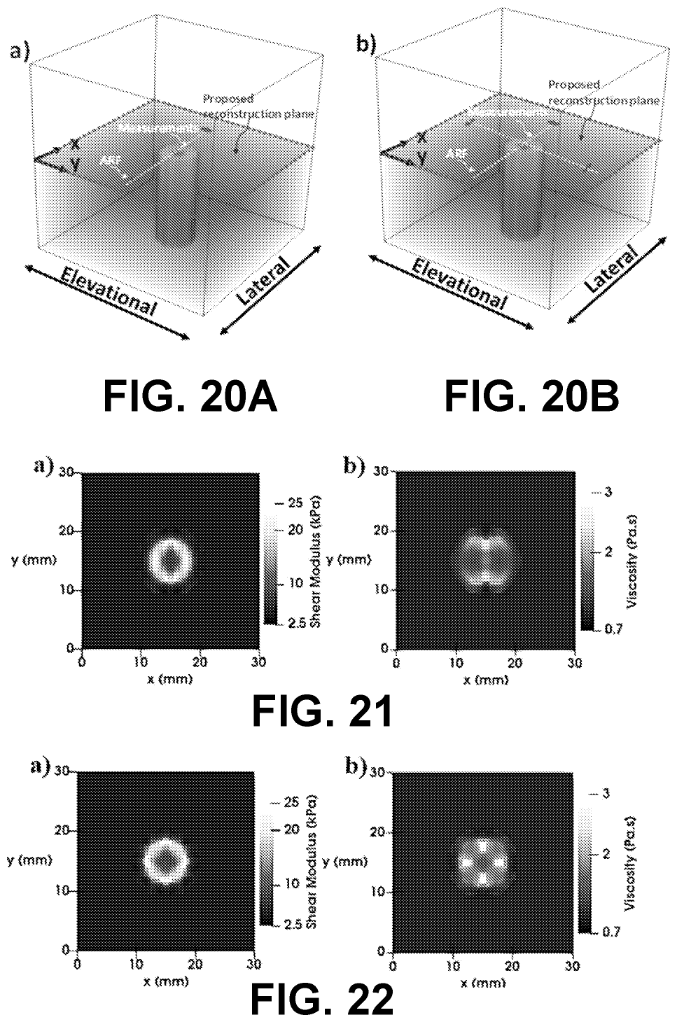

Reconstruction with multiple acquisitions. As seen in the examples in the previous section, using a single acquisition, promising reconstructed viscoelasticity maps were obtained for the homogeneous tumor case, whereas there are difficulties in recovering the viscoelasticity maps (especially viscosity) for heterogeneous tumors. In an attempt to improve the imaging, two examples were presented where a handful of different acquisitions are used with the hope of a better illumination of the target. Specifically, consider the pushes and acquisition planes shown in FIGS. 20A and 20B, which illustrate multi-acquisitions setup for two pushes and four pushes, respectively; one at a time. Note that pushes on opposite sides are utilized to maximize the illumination benefit of the additional push(es). FIG. 21 illustrates the reconstruction with the two-push configuration: plot (a) shows the reconstructed shear modulus map and plot (b) shows the reconstructed shear viscosity map. FIG. 21 shows the reconstructed viscoelasticity maps when using data obtained from two ARF applications (one at a time), one in the top and one in the bottom. Similarly, FIG. 22 illustrates the reconstruction with the four-push configuration: plot (a) shows the reconstructed shear modulus map and plot (b) shows the reconstructed shear viscosity map. FIG. 22 shows the reconstructed viscoelasticity for the same heterogeneous tumor but with two additional acquisitions (i.e., total of 4 pushes). Clearly, adding pushes significantly improved the reconstructed viscoelasticity maps, although the viscosity map still not as good as shear modulus map.

It should be emphasized that joint reconstruction of elasticity and viscosity is not a trivial task because of the strong dependence between the parameters (shear elasticity and viscosity), as well as the limited data availability. However, because the footprint of the viscosity, which is translated to data as amplitude attenuation, is very small compared to the footprint of the shear modulus, it is not unexpected to get a better reconstructed shear modulus map than that of viscosity. This is evident in almost all of the results presented earlier. Similar to this observation, it has been shown in the context of seismic full-waveform inversion that it is possible to reconstruct attenuation parameters (equivalent to viscosity) only if the signature of the viscosity in the data is strong enough. Motivated by this, it is proposed to first reconstruct both parameters jointly, with the expectation that viscosity map will not be as accurate as the shear modulus map. After this step is done, re-invert for viscosity only, while fixing the reconstructed shear modulus map. By applying this strategy on the previous example, it was possible to almost recover the true viscosity map. FIG. 23 shows the reconstruction of viscosity only after fixing the shear modulus: viscosity map.

A novel extension to the correlation-based full-waveform SWE to reconstruct high-resolution images of shear elasticity and viscosity has been presented. For homogeneous tumor-like tissues, it is possible to reconstruct an excellent shear elasticity and viscosity maps through multi-resolution with spatial averaging methodology. For the simpler homogeneous case shown in FIG. 15, only one push is sufficient; more pushes can improve the image further. It is difficult to reconstruct the high-resolution heterogeneous viscosity maps of soft tissue. Significant improvements were seen in the reconstructed shear modulus map utilizing the hierarchical approach presented in this work.

### Experimental Validation: Phantom Data Analyses and Reconstructions

It has been confirmed that changes in soft tissue stiffness could indicate the onset of a health disease. To this end, scientists have been developing methodologies for imaging soft tissue stiffness, including shear wave elastography (SWE), which infers the tissue stiffness map from examining the propagating characteristics of the shear waves generated from an acoustic radiation force (ARF). Several SWE methodologies have been proposed over the last decade; however, the majority of those methods are based on the local estimation of the shear wave velocity by calculating the time-of-flight (TOF), or the time-to-peak (TTP).

While the local methods provide an efficient solution for reconstructing the elasticity map of soft tissues, they require the availability of the motion in the entire region of interest (ROI). Given that ultrasound scanners detect motion in-plane only, both lead to limiting the reconstruction to 2D in-plane elasticity maps. Moreover, local methods ignore the 3D wave propagation effects, including complicated wave scattering for heterogeneous domains.

A framework for shear wave elastography based on correlation-based objective function and full-waveform modeling has been demonstrated that it is technically feasible to reconstruct a 2D elasticity map of tumor-like tissue utilizing only few measurements sampled on a line. The framework has been verified through several digital phantoms where the measurements were synthetically generated. By extrapolation, this could be a potential methodology for reconstructing 3D maps directly from 2D measurements acquired using conventional ultrasound scanners.

METHODS. In this section, the methodology of reconstructing the elasticity maps of two phantoms given the SWE data measured on a line. Beginning with the description of the materials used, the experimental setup and the acquisition protocol, the signal processing steps are then presented, and initial data analyses provided along with comparison between the measurements and simulated shear wave motions. Finally, the optimization framework is reviewed for the reconstruction.

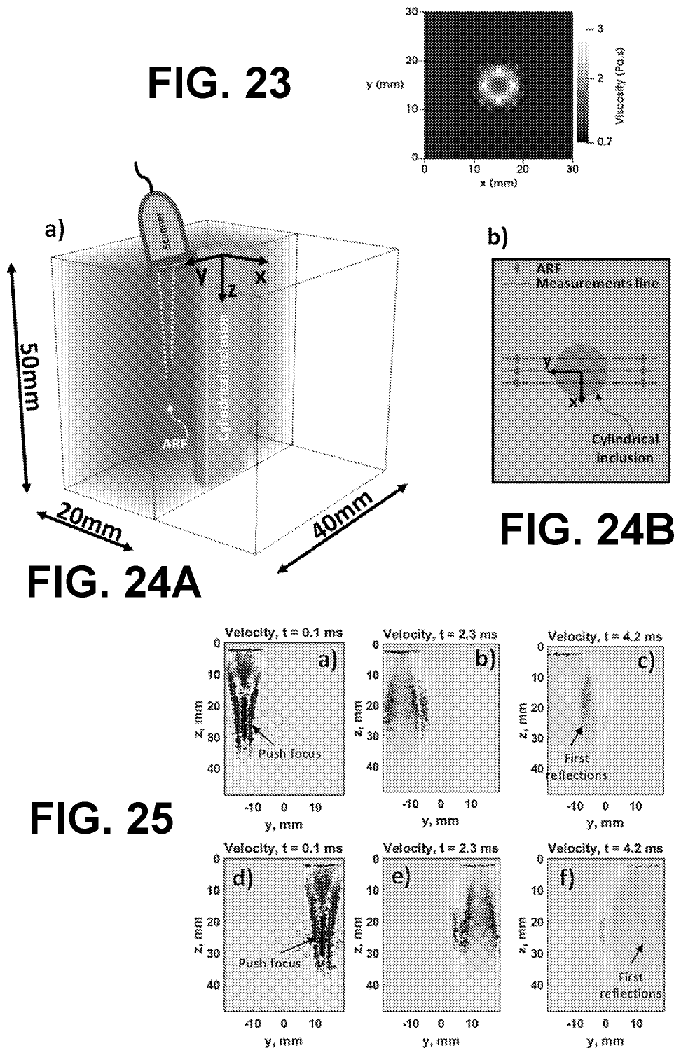

Materials, experimental setup and acquisition. The experiments were conducted on two phantoms, labeled phantoms I and II, with a cylindrical inclusion of a diameters of approximately 12 mm for phantom I, and 14 mm for phantom II. The configuration of the cylinder relative to SWE acquisition is illustrated in FIG. 24A, which is a 3D representation of the acquisition. FIG. 24B shows a setup projected on a 2D plane (x-y) passes through the push focus. In a 2D plane, the measurements plane becomes a line. The background is made of soft material whereas the inclusion is made of stiffer material.

Shear wave motion is generated by application of acoustic radiation force (ARF). Six Pushes were applied, one at a time; three on the left and three on the right of the inclusion, see FIG. 24B. For ARF application, the L7-4 linear array probe (Philips Healthcare, Andover, MA) with 128 elements and a center frequency of 5 MHz was used. One focused push beam (center frequency=4.1 MHz, F/1.5, focal depth=25.1 mm) was transmitted with a duration of 400 μs, and push line is at 6.5 mm and 31.5 mm. The ARF was configured such that it is about 5 mm from the border of the cylindrical inclusion. The push duration was 400 μs applied as follows: push for 200 μs then wait for 20 μs, then another push for 200 μs, finally wait for another 20 μs. The transducer was then switched to the imaging mode, where the first motion is detected after 240 μs (3×80 μs).

Plane Wave Compounding (PWC) was performed to track the shear wave propagation. The tracking beam had a center frequency of 5 MHz, and its Pulse Repetition Frequency (PRF) was 12.5 kHz. PWC excited all transducer elements at three angles [−4°, 0°, 4° ] without transmit apodization. The frame rate of PWC was increased to PRF by resorting to Time Aligned PWC (TAPWC) method to use interpolation of IQ data. The Matlab UltraSound Toolbox (MUST) is used for the receive beamforming (BF). To perform BF using the MUST, RF data acquired by the Verasonics system were saved in memory of the host computer. IQ data were converted from RF data and input to delay-and-sum (DAS) beamforming with receive F/#1.5 and a Hamming apodization. The transmit voltage was set at ±80 V and it applied to both the push beam and plane wave transmission for the measurement. To obtain motion data, the time delay between IQ data of two consecutive compounded images was calculated, which corresponds to the particle velocity profile. A 1D autocorrelation method (Kasai method) was used as a time delay estimation algorithm with the kernel size of 3λ. No filtering was applied to the motion data unless otherwise stated. The final motion data were 317×256 spatial pixels (with axial and lateral resolution of 149 μm along the y-axis, and 154 μm along z-axis) whereas the temporal sampling is 80 μs with total of 141 frames, leading to a total duration of detection of 11.28 ms.

Initial inspection of SW motion: FIG. 25 depicts different snapshots of the shear wave propagation in phantom I, measured on the middle plane. Snapshots (a-c) show ARF is on the left and (d-f) show ARF is on the right. Notice the width of the wavefront is increased just after the waves enter the cylinder. Also notice the location of the first reflection with respect to y axis in snapshots (c) and (f) of FIG. 25, which indicates that the cylinder may not be perfectly centered in the middle, and/or there are some imperfections in its cylindrical shapes (e.g., bumps).

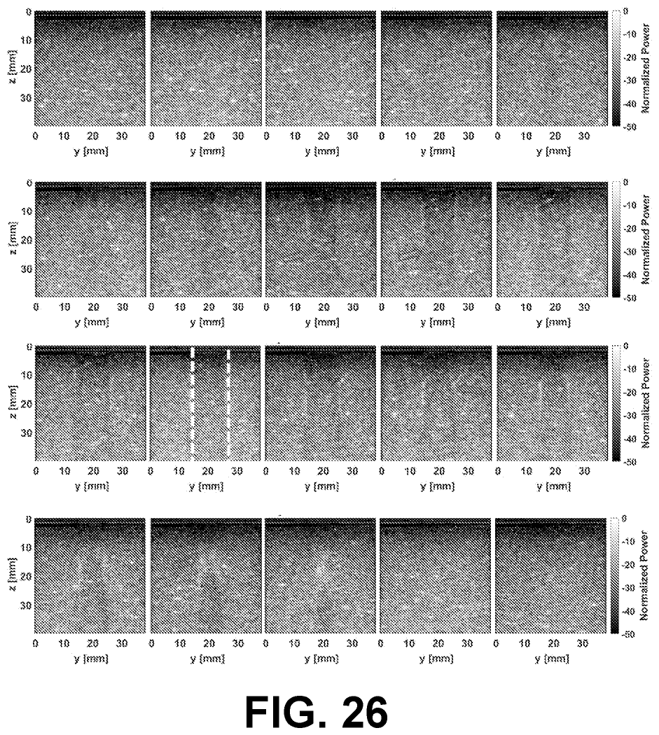

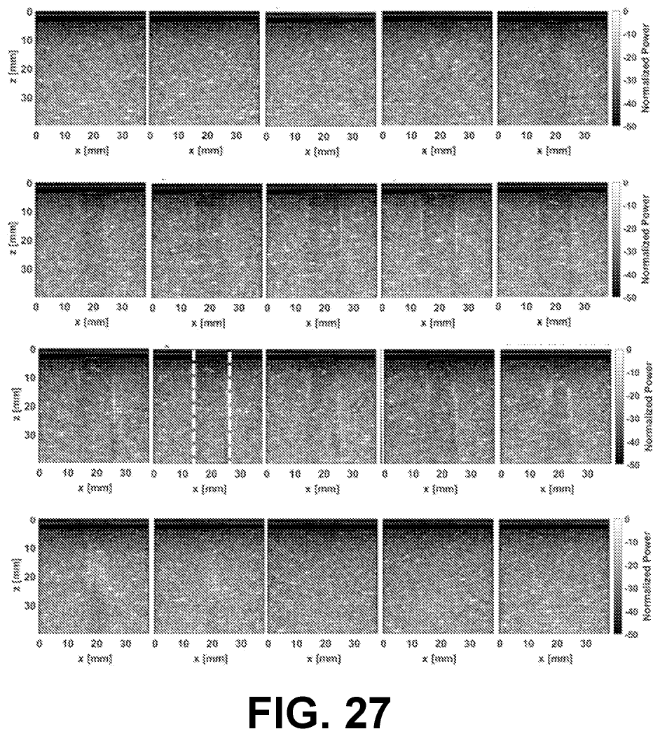

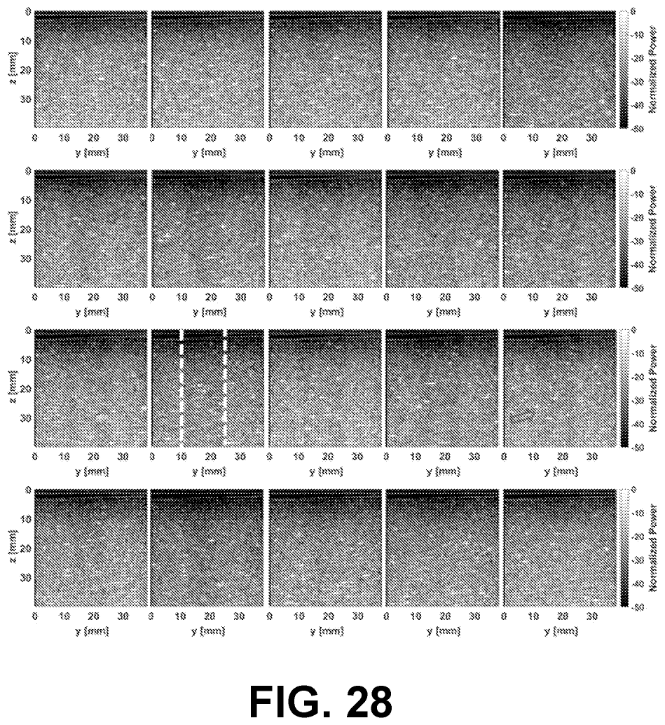

Inspecting B-mode images: B-mode imaging was performed to examine the actual geometry of the cylindrical inclusion inside the phantoms. For phantom I, two different orientations of B-mode imaging were performed, one is by imaging through the x-z plane, and another by imaging through the y-z plane. FIG. 26 shows B-mode images of phantom I acquired at 20 y-z planes. Each plane is 1 mm apart from the other. The imperfections are highlighted by arrows. Notice that the cylinder is not perfectly prismatic. The dashed-white lines indicate the location of the cylinder at a plane passing through its center. Notice that the cylinder is not centered in the middle with respect to the y-axis. Also note that, the diameter of the cylinder may not be exactly 12 mm. FIG. 27 shows B-mode images of phantom I acquired at 20 x-z planes. Each plane is 1 mm apart from the other. The imperfections are highlighted by arrows, notice that the cylinder is not perfectly prismatic. The dashed-white lines indicate the location of the cylinder at a plane passing through its center. Notice that the cylinder is almost in the middle centered with respect to the x-axis. For phantom II, B-mode imaging is carried out in the y-z plane only. FIG. 28 shows B-mode images of phantom II acquired at 20 y-z planes. Each plane is 1 mm apart from the other. The imperfections are highlighted by arrows, notice that the cylinder is not perfectly prismatic. The dashed-white lines indicate the location of the cylinder at a plane passing through its center. The estimated diameter is 14 mm. Notice that the cylinder is almost in the middle centered with respect to the y-axis. For each orientation, 21 planes with 1 mm apart were acquired, 11 of them are passing through the cylinder, and 5 planes are outside on each side. Upon examining the B-mode images that are depicted in FIGS. 26-28: first, both cylinders are not perfectly prismatic along the depth of the phantoms, second, the cylinders' diameters at the focal depth may not be exactly 12, 14 mm, for phantom I and II respectively. Finally, while the phantom I is almost centered in the middle of x-axis, there is an obvious shift with respect to y-axis. These 3 observations may result in artifacts in the reconstructed images. A comparison between the measured and simulated shear wave motions was conducted, where the simulations were carried out on the 2D and the cylinders which were assumed to be an infinitely long and perfectly circular. The resulting match was rather poor, consistent with the observations related to B-mode images.

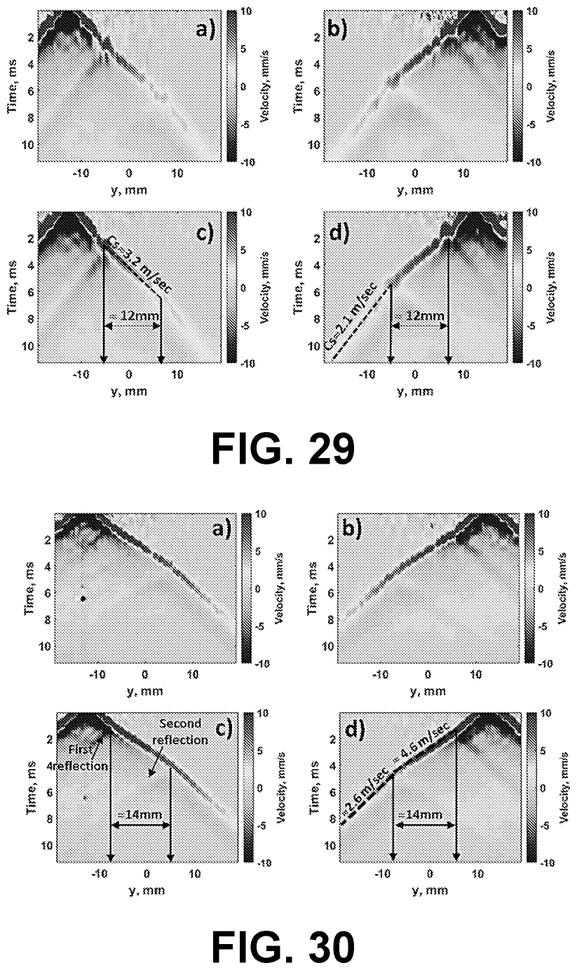

Signal processing and data analyses. The particle velocity measurements were made in time domain and in the vertical (y-z) plane, but the interest was in reconstructing an image in horizontal (x-y) plane (see FIG. 14B), where the acquisition plane is projected as a line on the 2D (x-y) plane. Approximating the push to be infinitely long in the z direction, the measurements on the entire y-z plane are reduced to one line of measurements, i.e., at a specific depth. The choice of the depth considered for the measurements could be arbitrarily, however, since the ARF profile is not prismatic, it was recommended that the measurements along the line that passes through the push focus, i.e., at z=25.1 mm. To enhance the signal-to-noise ratio, the depth was taken as the average of the measurements over 10 pixels along the z axis. FIGS. 29 and 30 depict shear wave motion measured for both phantoms at the midline, with and without depth averaging. FIG. 29 shows Phantom I—shear wave motions measured on the central plane at depth=25.1 mm: snapshots (a) and (b) are without depth averaging and snapshots (c) and (d) are with depth averaging over 10 pixels with the left column having the push on left, and the right column having the push on right. The estimated group velocity of the background and inclusion material could be calculated from the slopes of the wavefronts as indicated in the image. Also, arrows indicate the location of the cylinder w.r.t. y-axis. It can be seen that signal is clearer and smoother when performing depth averaging. FIG. 30 shows Phantom II—shear wave motions measured on the central plane at depth=25.1 mm: snapshots (a) and (b) are without depth averaging and snapshots (c) and (d) are with depth averaging over 10 pixels with the left column having the push on left, and the right column having the push on right. The estimated group velocity of the background and inclusion material could be calculated from the slopes of the wavefronts as indicated in the image. Also, arrows indicate the location of the cylinder w.r.t. y-axis.

Estimating initial mechanical properties: For both phantoms, the group velocities were examined in the inclusion and background, from the corresponding slopes of the wavefronts (see FIGS. 29 and 30). The estimated shear wave velocities of the inclusion and background material respectively are approximately 3.2 and 2.1 m/s for Phantom I, and 4.6 and 2.6 m/s for Phantom II. Therefore, the initial estimates of shear modulus are approximately 10.2 and 4.4 kPa for Phantom I, and 21 and 7 kPa for Phantom II.

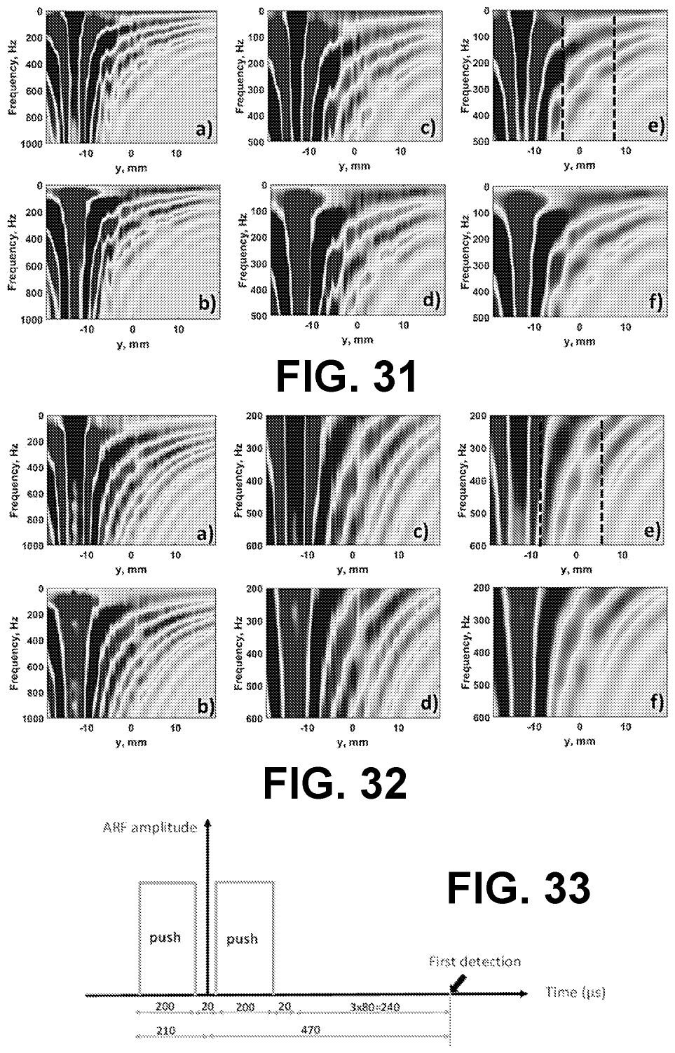

Transforming data to frequency domain: Since the framework is based on reconstruction in the frequency domain, the Fast Fourier Transform (FFT) can be used to transfer the time-domain measurements (y-t) to the frequency domain measurements in (y-f), where f is the frequency in Hz. Appropriate padding (specifically, increase the size of the time array to 2¹³) is utilized in the process. The Fourier transformed measurements for both phantoms are depicted in FIGS. 31 and 32. FIG. 31 illustrates the Fourier transformed data for phantom I where the push is on the left: snapshots (a) and (b) are the non-smoothed data spectrum up to 1000 HZ, snapshots (c) and (d) are the non-smoothed data spectrum up to 500 HZ, and snapshots (e) and (f) are the data spectrum up to 500 HZ smoothed by Gaussian filter of window size=20 pixels. The top row is the real part of the velocity, and the bottom row are the imaginary part. The black-dashed line indicates the location of the cylinder. Black-dotted lines indicate the estimated place of the cylinder. FIG. 32 illustrates the Fourier transformed data for phantom II where push is on the left: snapshots (a) and (b) are the non-smoothed data spectrum up to 1000 HZ, snapshots (c) and (d) are the non-smoothed data spectrum from 200 up to 600 HZ, and snapshots (e) and (f) are the data spectrum from 200 up to 600 HZ smoothed by Gaussian filter of window size=20 pixels. The top row is the real part of the velocity, and the bottom row are the imaginary part. The black-dashed line indicates the location of the cylinder. Black-dotted lines indicate the estimated place of the cylinder. While the entire frequency range would be ideal for reconstruction, since the signal is not uniform across the frequency range, we focus on frequency window with strong signals. This is performed through visual inspection (see (a) and (b) of FIG. 31 and (a) and (b) of FIG. 32); for phantom I, most of the energy are focused between 0 and 500 HZ. whereas for phantom II, energy is focused at 200-600 HZ. These data are shown in (c) and (d) of FIG. 31 and (c) and (d) of FIG. 32. There is clear evidence of noise in these signals, which are removed by applying Gaussian filter with a smoothing window of 20 pixels (in time and space e.g., y-t).

Time shift correction: Only one ultrasound transducer is used for the ARF application and detection. Therefore, there is an unavoidable time lag between the ARF application and first detection. While accounting for this is simple in the time domain, it becomes more involved in frequency domain, as both time shift and missing measurements are to be accounted for. The issue can be circumvented by simply examining the waves away from the source, where the (shear) wave is not expected to reach before the first measurement. Since the issue of missing measurement no longer applies, time shift of τ=510 μs can simply be performed by simple phase shift in frequency domain with e^{-iωτ}. FIG. 33 illustrates the push sequence and timing followed in SW generation and acquisition.

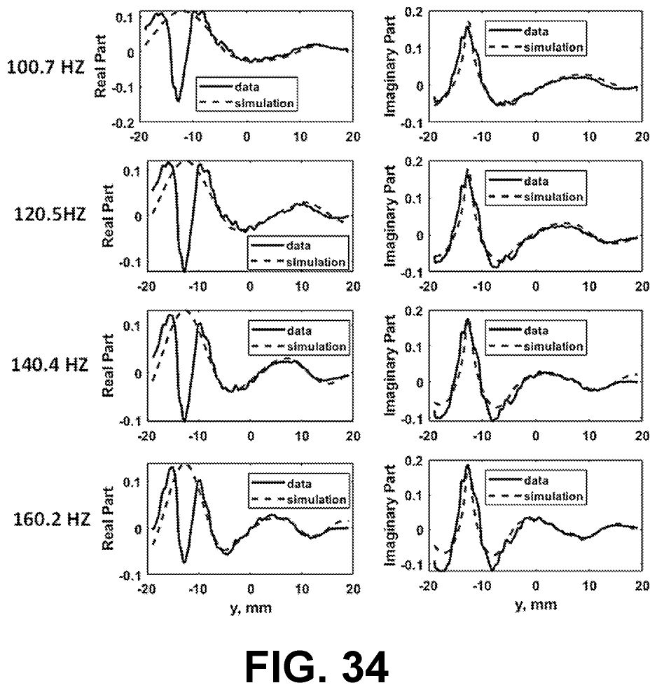

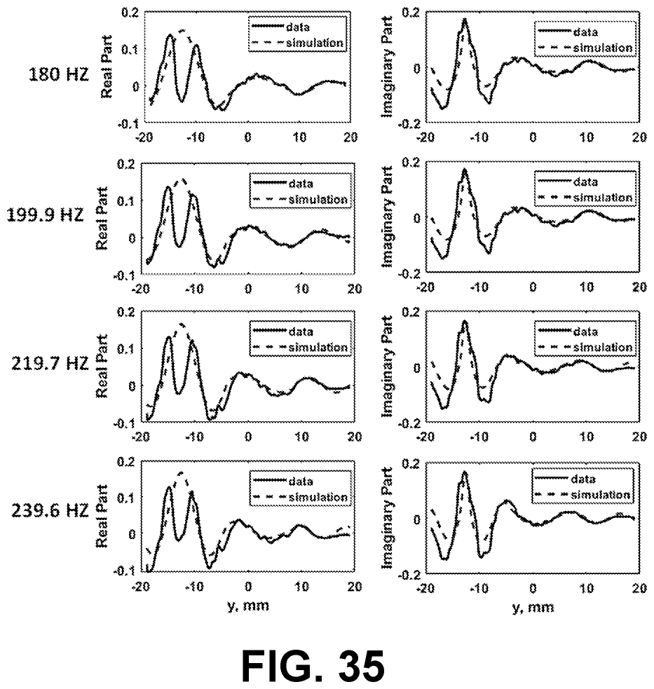

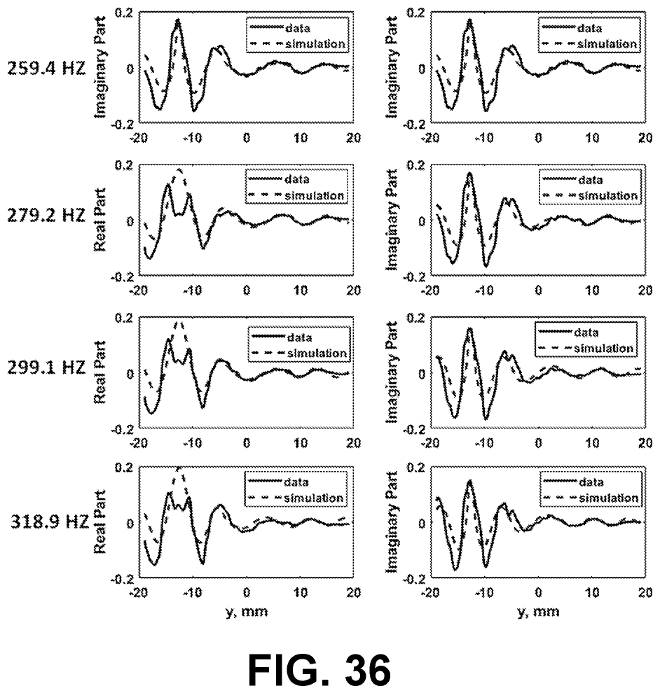

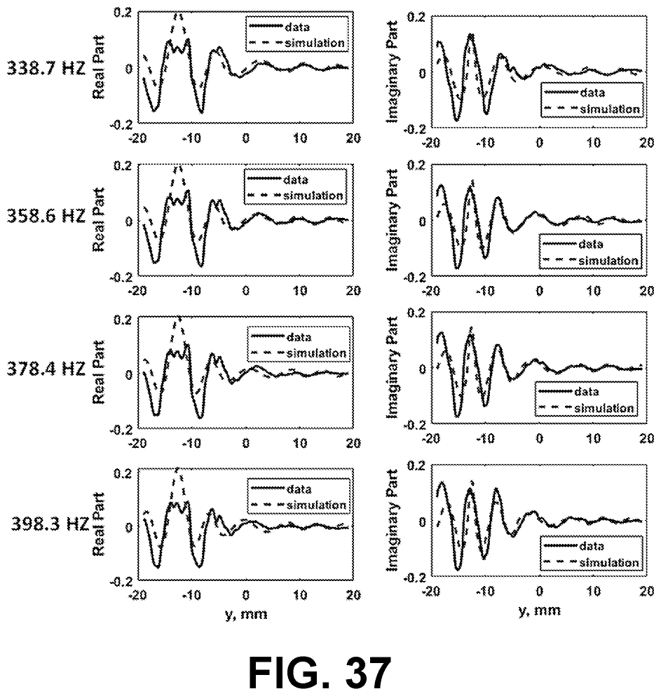

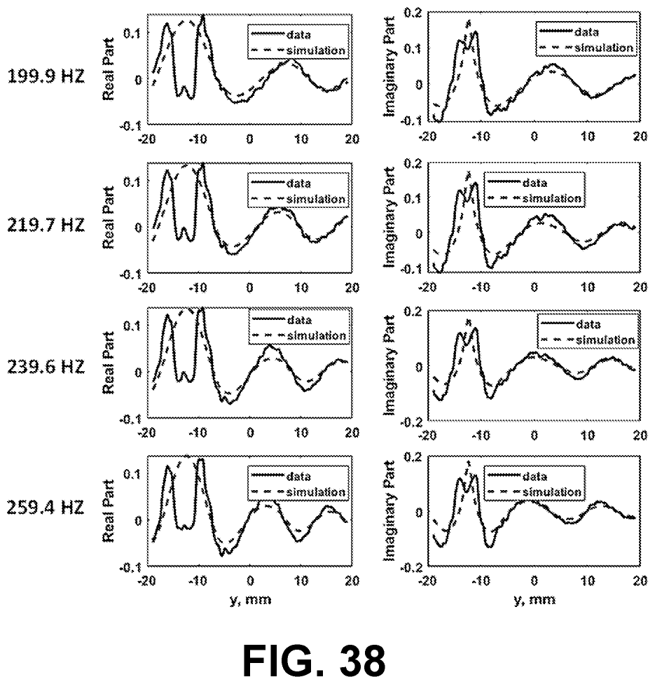

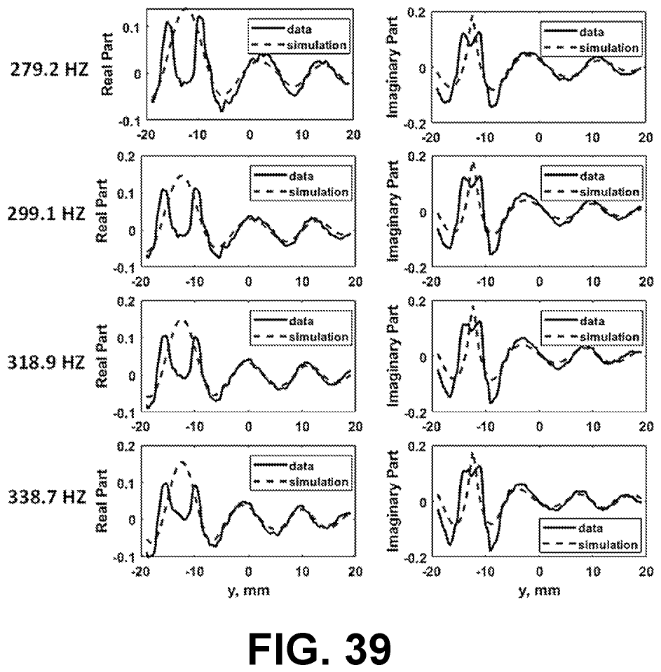

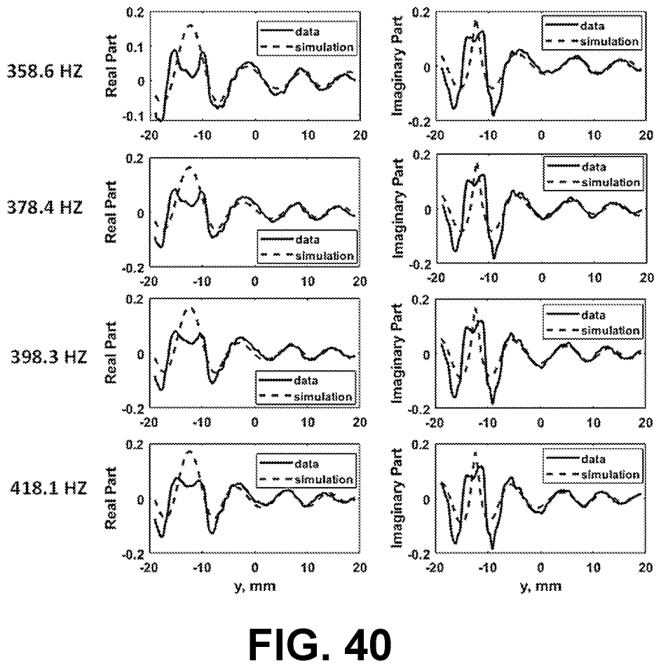

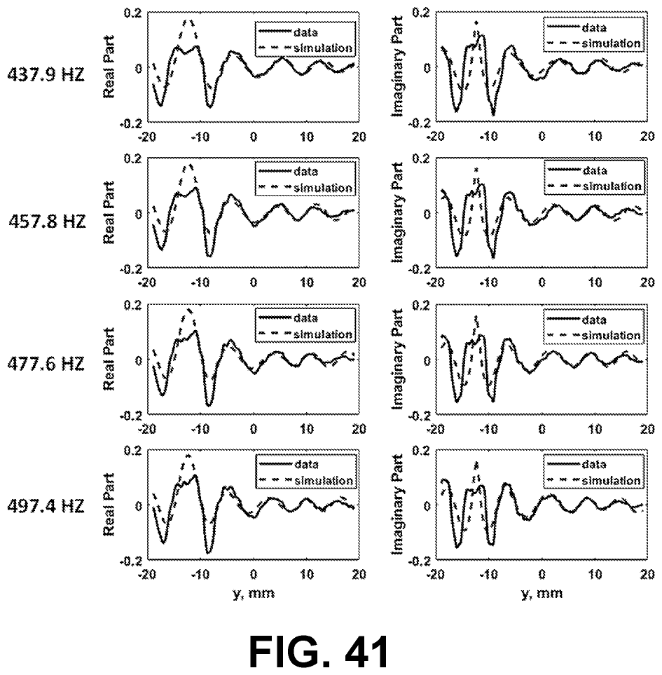

Comparison between simulated and measured motion: Estimates for the cylindrical inclusion geometry, mechanical properties, have been obtained and corrected for the time-shift. Before proceeding to the reconstruction step, it would be best to explore to what extent these estimates are accurate. This can be done through a comparison between the measured and simulated motions, where the mechanical and geometrical properties estimations are incorporated into the simulations (note that the measured quantities are particle velocities, whereas the primary unknowns in the simulation are the displacements. The simulated particle velocities are simply obtained by standard scaling in the frequency domain, (vₘ=iωuₘ), where vₘ is the measured SW velocity and uₘ is the corresponding SW displacements. These comparisons were performed for both phantoms over a range of frequencies as follows: Phantom I, from 100.7 up to 398.3 HZ with an increment of approximately 19.8 HZ, and for phantom II, from 199.9 up to 497.4 HZ, with the same frequency sampling. For Phantom I, the geometrical and mechanical properties previously estimated in addition to a viscosity of 0.1 Pa·s are used in the simulation, and we could see a good match between the simulation and measurements. However, for Phantom II, when using the estimated mechanical properties and geometries in the simulation, a poor match is obtained. Extensive sensitivity studies were carried out where the mechanical properties and geometry of the inclusion for Phantom II were varied. A good match was achieved using the following inputs: shear modulus of the background and inclusion 10 and 16 kPa, respectively, whereas the viscosity is set to zero and the diameter of the cylinder is 12 mm. FIGS. 34-41 detail these comparisons. FIG. 34 illustrates Phantom I comparison between the simulated and measured shear wave motion for 100.7-160.2 HZ. FIG. 35 illustrates Phantom I comparison between the simulated and measured shear wave motion for 180-240 HZ. FIG. 36 illustrates Phantom I comparison between the simulated and measured shear wave motion for 259.4-318.9 HZ. FIG. 37 illustrates Phantom I comparison between the simulated and measured shear wave motion for 338.7-398.3 HZ. FIG. 38 illustrates Phantom II comparison between the simulated and measured shear wave motion for 199.9-259.4 HZ. FIG. 39 illustrates Phantom II comparison between the simulated and measured shear wave motion for 279.2-338.7 HZ. FIG. 40 illustrates Phantom II comparison between the simulated and measured shear wave motion for 358.6-418.1 HZ. FIG. 41 illustrates Phantom II comparison between the simulated and measured shear wave motion for 437.9-497.4 HZ.

Examining FIGS. 34-41, a good match exists between the experimental measurements and the simulation except in the region where the ARF is applied. This is expected since the simulation is performed in two-dimensional space, where 3D ARF is being modeled as a 2D sine-square pulse. Therefore, when reconstructing the elasticity maps, it is recommended not to consider the data around the region of the ARF application, e.g., for both phantoms, we only consider data from y=−5 mm up to y=15 mm. Moreover, it is noticed that the match between the simulation and experimental measurements is reduced at higher frequencies for phantom I, especially starting from 259.4 HZ up to 398.3 HZ, see FIGS. 36-37. This is expected too, since the simulation implicitly assumes that the cylinder is infinite and prismatic, however, when inspecting the B-mode images, the actual cylinder is not perfectly circular, and not prismatic, even it is not infinitely long. Therefore, at higher frequencies, waves “see” the geometric approximations, leading to this mismatch at higher frequencies. On the other hand, for Phantom II, the match seems to be worse at lower frequencies. It should be emphasized here that these comparisons should be used only for preliminary guidance, as they are based on rather crude estimates of the mechanical properties of the phantom, combined with ARF idealizations.

RESULTS. The reconstruction with measurements from one, two and three SWE measurement planes (from three different ARF pushes) was investigated. In addition, the effect of parameterization was investigated and two different methods of parameterization, the piecewise multi-constants and piecewise bilinear tried. Images at two resolutions were reconstructed, specifically, resolution 1 from 100.7 to 160.2 HZ and resolution 2 from 100.7 to 239.6 HZ for Phantom I, and resolution 1 from 199.9 to 279.2 HZ and resolution 2 from 199.9 to 378.4 HZ for Phantom II, both at sampling rate of 19.8 HZ. The reconstruction can be started by setting the shear modulus to the constant value equal to background modulus, i.e., 4.4 kPa for Phantom I and 8 kPa for Phantom II. Only the axial (z) component of the SW is considered for reconstruction. Note that no regularization terms were used in the reconstruction framework. Additionally, multi-constant function was initially used for the parameterization. These two resulted in non-smoothed inversion. To smooth the inversion, we applied a Gaussian filter with window of 10 pixels (5 mm).

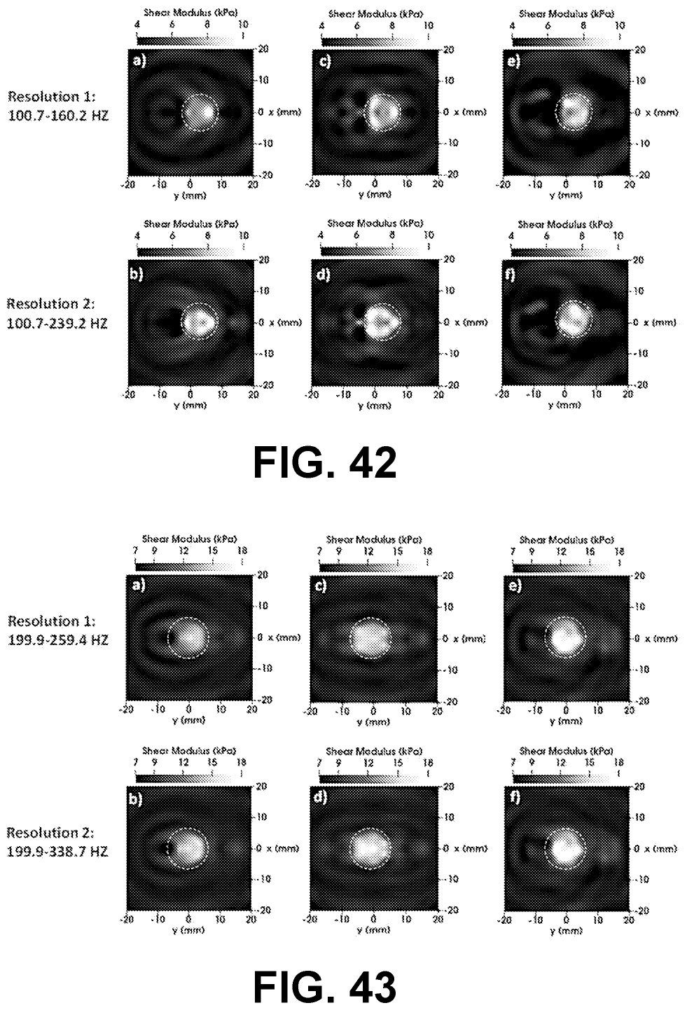

Reconstruction with piecewise multi-constants parameterization. The reconstructed shear modulus maps are presented in FIGS. 42 and 43, for Phantom I and II, respectively. FIG. 42 shows reconstructed shear modulus maps for Phantom I: with (a) and (b) using single push on the left, (c) and (d) using two-push, one on the left and one on the right, (e) and (f) using three-push on the left. The white-dotted line represents the expected inclusion of 12 mm diameter. For Phantom I, the images when using three pushes on the left are closer to expectations. In terms of the value of the shear modulus, all the images are within the expected shear modulus values, with (f) of FIG. 42 being the closest to the expected values and shape. Note that the main benefit of the proposed method is imaging; group velocity-based estimates only work because of the homogeneous nature of the phantoms.

FIG. 43 shows reconstructed shear modulus maps for Phantom II: with (a) and (b) using single push on the left, (c) and (d) using two-push, one on the left and one on the right, (e) and (f) using three-push on the left. The white-dotted line represents the expected inclusion of 14 mm diameter. Interestingly, for this phantom, the reconstruction seems better even for the single push configuration; multi-resolution reconstruction does not add much to the images (compare the first and second rows in FIG. 43).

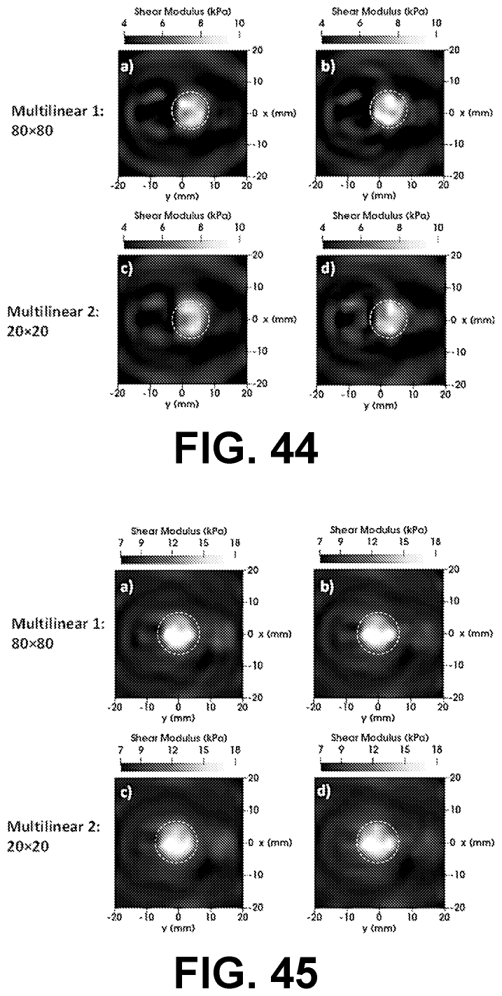

FIG. 44 shows reconstruction with multi-linear parameterization for Phantom I with (a) and (c) having resolution 1 (i.e., 100.6-160.2 HZ), and (b) and (d) having resolution 2 (i.e., 100.6-239.7 HZ). The multi-linear parameterization with 80×80 parameters (first row), and 20×20 (second row). The white-dotted line represents the expected inclusion of 12 mm diameter. FIG. 45 shows reconstruction with multi-linear parameterization for Phantom II with (a) and (c) having resolution 1 (i.e., 199.9-279.2 HZ), and (c) and (d) having resolution 2 (i.e., 199.9-378.4 HZ). The multi-linear parameterization with 80×80 parameters (first row), and 20×20 (second row). The white-dotted line represents the expected inclusion of 14 mm diameter.

Reconstruction with piecewise bilinear parameterization. As mentioned before, representing the shear modulus as multi-linear functions may result in better reconstruction, especially in the absence of regularization term. Motivated by this, further inversion was carried out where the piecewise bilinear parametrization was utilized with 20×20 and 80×80 to represent the shear modulus map; this is done for both phantoms. In this section, only 3 pushes on the left was considered.

For Phantom I, the benefits of using bilinear parameterization with 80×80 parameters is not clear. In fact, when using lower-resolution parameterization (20×20), there is downgrade in the images for phantom I. On the other hand, for Phantom II, there are some changes in the reconstructed images when using the bilinear with the same parameter resolution.

A validation study for the developed shear wave elastography framework has been presented for reconstructing shear modulus map of soft tissues. The framework was built on full-waveform inversion with a cross-correlation misfit functional. Measurements were sparse and sampled on a line (in this work the maximum is 3 lines), which represents the acquisition plane but projected onto a line in the (x-y) plane. Two tissue-mimicking phantoms with different mechanical and geometrical properties were used. In both phantoms, there is a stiffer cylindrical inclusion where the axis of the cylinder is in the z direction. The goal is to reconstruct the shear modulus map of both phantoms for a given depth. Different number of pushes were investigated for reconstruction, as well as two different parameterizations, piecewise constant and piecewise bilinear. While both single and multi-push inversion worked for one of the phantoms, multi-push inversion helped enhance the image for the other phantom.

Three-Dimensional Reconstruction of Soft Tissue Viscoelasticity from Planar Shear Wave Elastography Measurements

Shear wave elastography (SWE) uses ultrasound to image soft tissue's mechanical properties. Most of the SWE methods are based on the local estimation of the velocity of shear wave that is induced internally (acoustic radiation force, ARF), or externally (mechanical vibrator). To date, all SWE modalities use ultrafast ultrasound frames to detect the shear wave motions in the tissues. Colored maps of the soft tissue mechanical properties are then reconstructed, which provides useful information needed for disease diagnosis. This technique has been clinically applied for imaging different organs, including breast, liver, prostate, among others.

The mainstream of SWE technologies are limited to reconstructing 2D maps of the tissue mechanical properties. They do not account for the complex wave physics in 3D, and do not provide a complete picture of the 3D tissue mechanical properties. Alternatively, to reconstruct a 3D map of the tissue, many 2D maps are reconstructed and stacked together, which requires several acquisitions. Unfortunately, these too do not truly account for the full wave scattering effects in 3D. Therefore, it would be of importance to develop a method that could reconstruct 3D maps of tissues' mechanical properties utilizing the well-established conventional US devices, while not overly idealizing the wave physics.

Motivated by these, reconstruction of 3D maps of tissue's mechanical properties from SW motion detected by conventional ultrasound is proposed. Specifically, it is demonstrated through synthetically generated SW motions that it is technically feasible to reconstruct a 3D elasticity map of tumor-like tissue from a single acquisition in settings like those of the conventional ultrasound imaging. Recently, it has been demonstrated through in-silico and phantoms experiments that it is feasible to reconstruct 2D elasticity maps from 1D SW measurements. The methods can be extended to 3D, i.e., use SWE measurement on a 2D plane, from an acoustic radiation force to reconstruct 3D images of mechanical properties. This can be done by combining full-wave simulation in 3D, combined with a PDE-constrained optimization framework with cross-correlation objective function and a multi-resolution approach to solving the optimization problem. The methodology was verified through two different in silico experiments, where tumors of different sizes were successfully reconstructed to a good accuracy.

METHODS. In this section, details of the new methodology are presented starting with the modeling of the shear wave propagation in a nearly incompressible viscoelastic soft tissue. Then the optimization framework is reviewed.

Forward model. Generally, waves amplitude attenuates because of the viscous nature of human soft tissue. Therefore, it is important to account for viscosity even if only elasticity is being reconstructed. Moreover, soft tissues are generally incompressible. Thus, in this work, the soft tissue can be modeled as an isotropic, incompressible, linearly viscoelastic medium. The assumption of linearity follows the fact that ARF is generally very low in amplitude that makes the material nonlinearity does not enter into picture. In addition, any change in the material properties that may result from the change in the fluid pressure after the application of the ARF were ignored. The partial differential equation (PDE) that describes 3D wave propagation in an isotropic incompressible viscoelastic medium induced by an ARF is as follows:

$$- ∇ \cdot σ ( \underline{x} , t ) + ρ \ddot{u} ( \underline{x} , t ) = f ( \underline{x} , t ) δ ( \underline{x} - {\underline{x}}_{o} ) , \underline{x} ∈ {Ω}_{ROI} ∈ {R}^{3} , \qquad ( 3.1 )$$

where σ is the 3×3 second order Cauchy stress tensor, f represent the acoustic radiation force (ARF), δ is Dirac function, x is the position vector in (x, y, z), xₒ is the position of the ARF, ρ is the tissue density (ρ=1000 kg/m³, e.g., water) and ü is the particle acceleration (vector field).

In this work, Kelvin-Voigt constitutive viscoelastic model is used as the underlying constitutive model to describe the behavior of the soft tissue. However, note that the method is general and other viscoelastic models could be utilized. Therefore, the stress-strain relationship is then written as:

$$σ = {σ}_{e} + {σ}_{v} = B ( G ) ε + C ( η ) \dot{ε} , \qquad ( 3.2 )$$

where (σₑ, σᵥ) are the elastic and viscous parts of the stresses respectively, B is the 6×6 fourth order shear elasticity matrix, C is the 6×6 fourth order shear viscosity matrix, G (kPa) is the shear modulus, η (Pa·s) is the shear viscosity, ε is the 3×3 second order strain tensor, and \dot{ε}=∂ε/∂t is the strain rate.

In this study, Equation (3.1) was solved numerically in the frequency domain so to have better control on the numerical dispersion resulting from the numerical discretization especially at higher frequencies. In addition, the solution process would be computationally more efficient if a proper set of frequencies were chosen. Finally, expanding frequency windows were utilized to do multiresolution imaging, which necessitates frequency domain forward modeling. Thus, Equation (3.1) was transformed from the time domain to the frequency domain, yielding the following equation.

$$∇ \cdot \overline{σ} ( \underline{x} , ω ) + \overline{f} ( \underline{x} , ω ) δ ( \underline{x} - {\underline{x}}_{o} ) = - {ρω}^{2} \overline{u} ( \underline{x} , ω ) , \underline{x} ∈ {Ω}_{ROI} ∈ {R}^{3} , , \qquad ( 3.3 )$$

where ω is the angular frequency and (σ̅, f̅, ū) are Fourier transforms of Cauchy stress tensor, ARF, and particle displacements. Further assume that the tumor is small compared to the homogeneous ROI and far from the surface. Given these assumptions, consider the background to be homogenous and waves travel outside the ROI without being reflected back into the ROI. Therefore, utilize a radiation boundary condition on the boundary of the ROI (∂Ω), which can be formally written as,

$$\overline{σ} \cdot n = Λ \overline{u} , \qquad ( 3.4 )$$

where n is the unit normal to the boundary surface ∂Ω and Λ is the Dirichlet-to-Neumann operator representing an absorbing boundary condition. While efficient versions of Λ would make the absorption more accurate (e.g., PML, PMDL), the simpler Sommerfeld radiation boundary condition was transformed.

As mentioned earlier, ARF amplitude is very low, thus soft tissue exhibits small deformations after ARF application. Therefore, consider the small deformation theory where,

$$ε = \frac{1}{2} ( ∇ \overline{u} + ∇ {\overline{u}}^{T} ) . , \qquad ( 3.5 )$$

The complete strong form of the PDE in the frequency domain can be obtained by combining Equations (3.2), (3.3), (3.4) and (3.5).

Finite element modeling: The strong-form discussed in the previous equation can be converted to weak form, leading to:

$$( v , \overline{u} ) = {\int }_{{Ω}_{ROI}} \overline{σ} ( \overline{u} ) : ε ( v ) - {\int }_{\partial Ω} ( \overline{σ} ( \overline{u} ) \cdot n ) v + {ρω}^{2} {\int }_{{Ω}_{ROI}} \overline{u} v + {\int }_{{Ω}_{ROI}} \overline{f} δ ( \underline{x} - {\underline{x}}_{o} ) v = 0 , , \qquad ( 3.6 )$$

$$∀ v ∈ V := \{ v : v ∈ {H}_{0}^{1} ( {Ω}_{ROI} ) \}$$

Equation (3.6) is the variational weak form of the strong PDE in Equation (3.3). The solution for Equation (3.3) is the vector field ū (the wavefield displacements in 3D) at which the residual [symbol] (ū)=0 (i.e., the residual vanishes). In this work, the finite element method (FEM) was used to solve Equation (3.3). Specifically, the Multi-physics Object Oriented Simulation Environment (MOOSE) was used, where the first term in the residual is modeled using the stress divergence kernel, the second term is the traction boundary conditions, the third term is modeled using reaction kernel and the last term is modeled using the Dirac kernel. Further, the 8-node hexahedron finite element was adopted with linear Lagrange shape functions for modeling the entire 3D ROI.

As mentioned earlier, most of the soft tissues are nearly incompressible (i.e., v→0.5). When using FEM with the traditional linear shape functions along with the incompressibility condition, the linear finite elements exhibit the phenomena of volumetric locking (also called shear locking), leading to incorrect deformations. To overcome this issue, one can use higher order finite elements, which generally leads to higher computational cost. However, there are several methods that have been proposed to correct for the volumetric locking associated with these traditional linear elements. In this work, the B-bar method was adopted, which is implemented in MOOSE. In this method, the total strain is decomposed into volumetric and deviatoric components, and then the volumetric component is approximated by the volumetric average of the strain over the element.

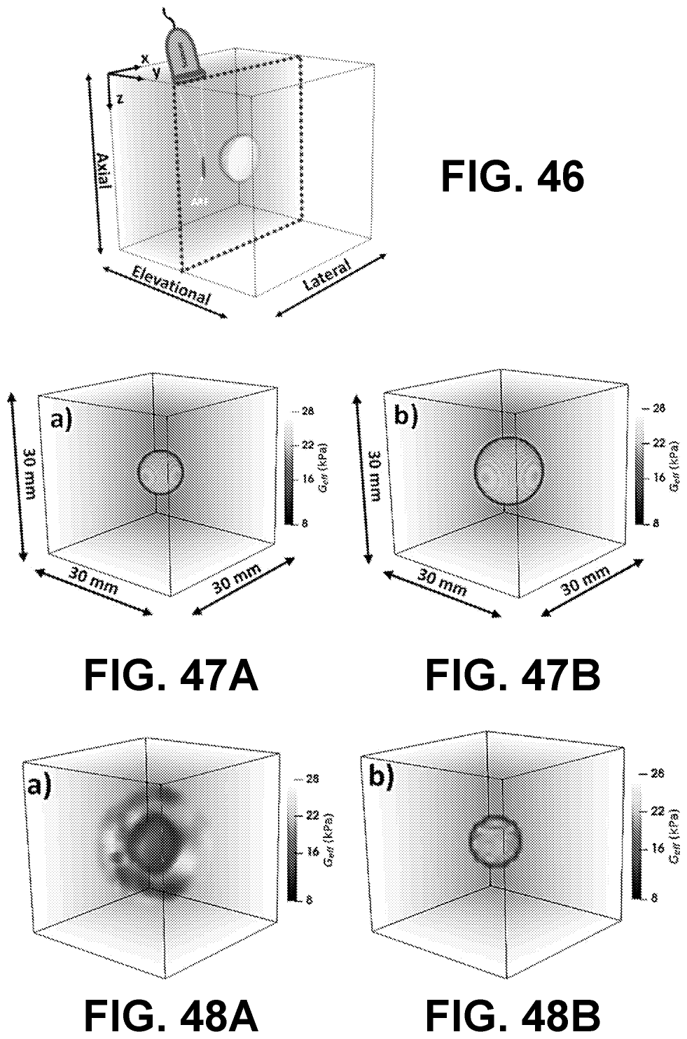

Reconstruction. The goal of image reconstruction is to infer physical parameters (e.g., the distribution of shear modulus and viscosity) by minimizing a misfit functional (e.g., objective functional [symbol] (p)) that measure the mismatch between the measurements and predicted (simulated) responses. In this work, the PDE-constrained optimization framework was adopted and an objective functional that is based on the cross-correlation (CC) between the measurements and predicted wavefields iteratively minimized. The major equations that describe the reconstruction framework are briefly highlighted. The PDE-constrained optimization problem can be written as:

$$\hat{p} = \begin{matrix}arg min \\ p\end{matrix} ( p ) , \qquad ( 3.7 )$$

$$Subjected to ( \overline{u} , p ) = 0$$

where p is the model parameter vector and \hat{p} is the optimal model parameter that minimizes the objective function, [symbol] (p) is the CC-functional which can be written as follows:

$$( p ) = 1 - \frac{{\vert d \cdot ( \overline{u} ( p ) ) \vert}^{2}}{{( ‖ d ‖ ‖ \overline{u} ( p ) ‖ )}^{2}} , \qquad ( 3.8 )$$

where d is the measurements ∈Ω_{data}⊆Ω_{ROI}, ū is the simulated wavefield and [symbol] is a projection operator that restricts ū to the measurement plane. FIG. 46 illustrates a conventional ultrasound shear wave imaging setup, the dotted rectangular plane represents the imaging plane often acquired in conventional ultrasound imaging. The dotted lines represent the acoustic beams used for ARF focusing.

As mentioned earlier, the Kelvin-Voigt model was utilized to represent the viscoelastic behavior of the medium:

$$\overline{G} = G + i ωη = {G}_{eff} ( 1 + \frac{i ω}{\overline{ω}} ) , \qquad ( 3.9 )$$

here, consider the constant relaxation time, and aim to reconstruct the G_{eff} that encapsulates the shear elasticity and viscosity values, therefore, p=G_{eff}. In this study, consider ω̅=15,000 rad/sec, which translates to a relaxation time of 0.067 ms. Since the number of pixels for shear modulus map is large, the adjoint formulation was adopted for gradient computation, where the gradient of cost functional w.r.t the effective shear modulus distribution i.e., g(G_{eff}) is given by:

$$g ( {G}_{eff} ) = - {λ}^{T} \frac{\partial ( u , {G}_{eff} )}{\partial {G}_{eff}} , \qquad ( 3.1 )$$

$$where ,$$

$${λ}^{T} \frac{\partial ( u , {G}_{eff} )}{\partial {G}_{eff}} = {\int }_{{Ω}_{ROI}} {σ}^{′} ( u ) : ε ( {λ}^{T} ) , \qquad ( 3.11 )$$

$${σ}^{′} ( u ) = \frac{\partial D}{\partial {G}_{eff}} ε ( u )$$

and λ, the adjoint variable, can be obtained by solving the adjoint problem as follows:

$${( \frac{\partial ( u , {G}_{eff} )}{\partial u} )}^{T} λ = {[ \frac{\partial ( {G}_{eff} )}{\partial u} ]}^{T} , \qquad ( 3.12 )$$

In the above equations, [symbol] (u, G_{eff}) is the residual resulted from finite element discretization, and T is the conjugate transpose operator.

There are several gradient-based optimization algorithms that could be used to solve the PDE constrained optimization problem. Based on our experience with seismic and shear wave elastography imaging problems, BFGS algorithm was utilized, which turned out to perform well for the numerical examples explored in this work.

Parameterization: Consider using the piecewise constant functions to represent the effective shear modulus distribution in 3D, where G_{eff}(x)=h₀(x)b(x), where G_{eff}(x) is the effective shear modulus distribution in 3D, b(x) is a function contains the values of shear modulus at user-defined coordinates, h₀(x) is a one-to-one mapping function to map the shear modulus values to the finite elements.

Restricting inversion to a window: Reconstructing the whole region of interest (ROI) would be of interest in some cases, however, in majority of the cases, the reconstruction is only necessary for a particular target (part of the ROI), e.g., tumor. The expectation is that the mechanical properties of background are usually known or can be easily determined using point SWE algorithms. In this work, the reconstruction can be restricted to a window that is smaller than the ROI, leading to a reduction in the number of parameters. This, in fact, would make the optimization process less ill-posed, and make it more possible to use fewer number of acquisitions. The size of the reconstruction window can be then initially determined by examining the B-mode images. However, given the uncertainty associated with B-mode images, the reconstruction was limited to a spherical window slightly larger than the actual inclusion. Finally, perform multiresolution imaging, first reconstructing with lower resolution using low-frequency data, followed by refining the image by including higher frequency data.

RESULTS. In this section, results of the study to reconstruct 3D maps of soft tissue's mechanical properties are presented using the setup in FIG. 46. Specifically, results to reconstruct 3D maps of the effective shear modulus for two spherical inclusions of 8- and 12-mm diameters, respectively. Furthermore, the robustness of the methodology was tested against noisy measurements by adding different Gaussian noise levels to the synthetic SW motions. The selection of frequency content for reconstruction is made based on the size of the tumor and that it is similar to the expected frequency content from real SWE scenarios. For all examples, synthetic measurements are acquired from a single acquisition, on the y-z plane. Measurements are spatially sampled every 1 mm, and only the axial component of the motions (along z-axis) were considered. In addition, the measurements are at least 2 mm far from the ARF focus, totaling 713 data points. The initial guess for all the tests is homogenous ROI with G_{eff}=25 kPa. For all the examples, the reconstructions were stopped after 40 iterations.

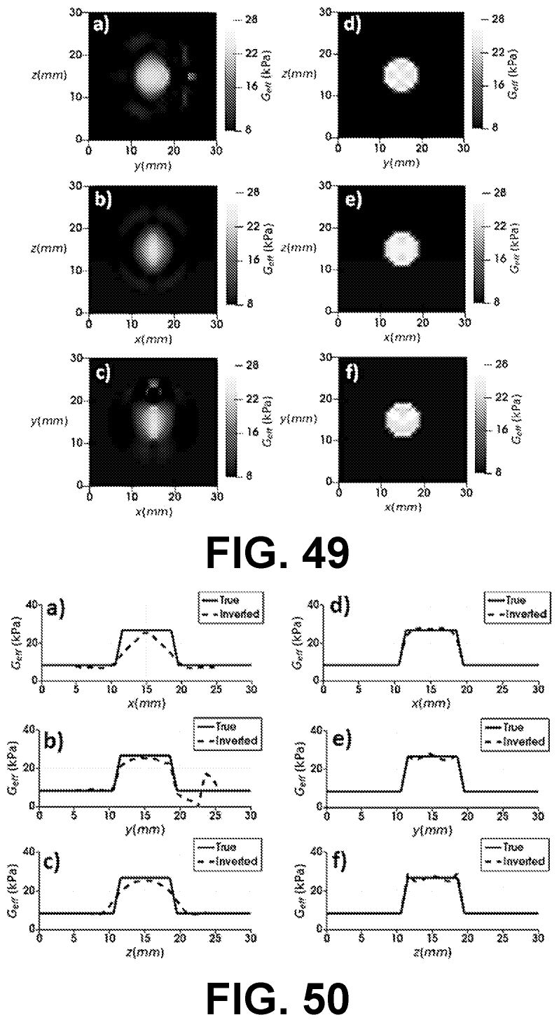

Reconstruction without noise. The first test involves reconstructing the 3D map of the effective shear modulus of an 8 mm inclusion utilizing noise-free SW measurements. FIGS. 47A and 47B illustrate the true effective shear modulus distribution for tumors of two different diameters: 8 mm, and 12 mm, respectively. The reconstruction is carried out in two resolutions, first, from 100-300 Hz, then from 100-500 Hz, both with sampling frequency of 50 Hz. As mentioned before, the initial guess of the effective shear modulus map is assumed to be homogenous with a value of 25 kPa. For the first resolution, assume no prior information about the size of the tumor, thus, the reconstruction was initially restricted to a spherical window of a diameter 20 mm. Once the image of the first resolution is reconstructed, the size of the reconstructed tumor was evaluated, and this information used as the prior information for another round of reconstruction, with refined parameter mesh and higher frequency data (resolution 2). To overcome the nonphysical values reconstructed during the reconstruction for resolution 1, the reconstructed modulus was spatially averaged. FIGS. 48A and 48B depicts the reconstructed 3D maps for the 8 mm tumor at resolution 1 (100-300 Hz) and 2 (100-500 Hz), respectively, where the initial reconstruction window can be seen in FIG. 48A, and the final 3D tumor image in FIG. 48B. FIGS. 49 and 50 depict 2D slices and 1D sectional profile of the 3D reconstructed maps. FIG. 49 shows the reconstructed 2D views of the effective shear modulus for the 8 mm tumor: for (a-c) resolution 1 (100-300 Hz), and (d-f) resolution 2 (100-500 Hz). FIG. 50 shows the comparison between the reconstructed and true effective shear modulus for the 8 mm tumor along x-, y-, z-axes that passes through the center of the tumor: for (a-c) resolution 1 (100-300 Hz), and (d-f) resolution 2 (100-500 Hz). There is excellent agreement between the true and reconstructed modulus maps.

Reconstruction with noise. This section focuses on reconstructing the effective shear modulus maps of a bigger tumor, (12 mm diameter), while the measurements are noisy. The noise levels are categorized into three categories based on the signal-to-ratio (SNR), specifically, low SNR=10, moderate SNR=20 and high SNR=30 dB. Since the tumor diameter is 1.5 larger than the one in the previous section, we just limited the maximum frequency to 400 Hz.

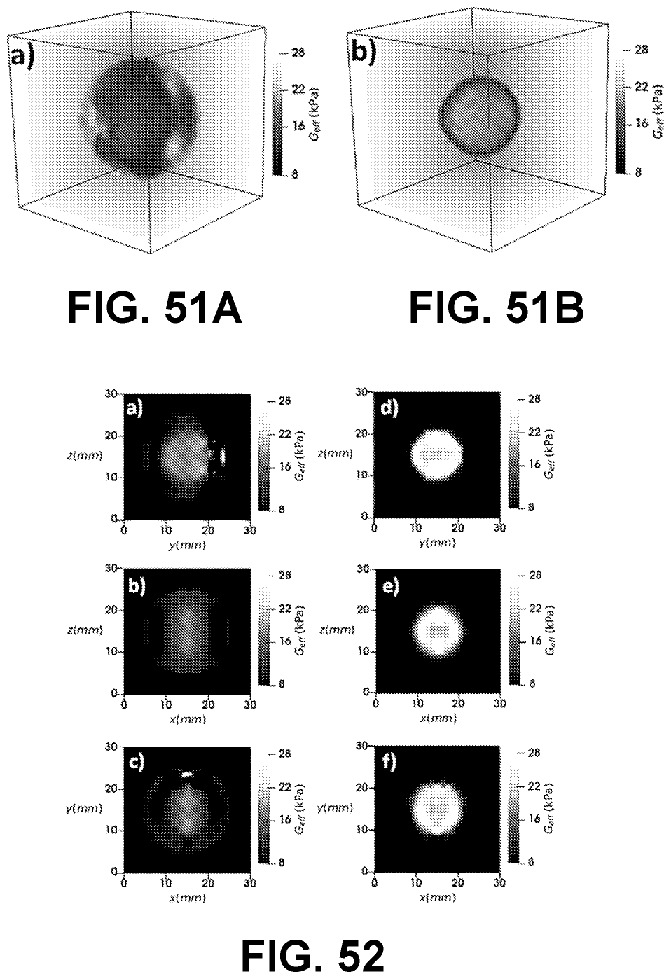

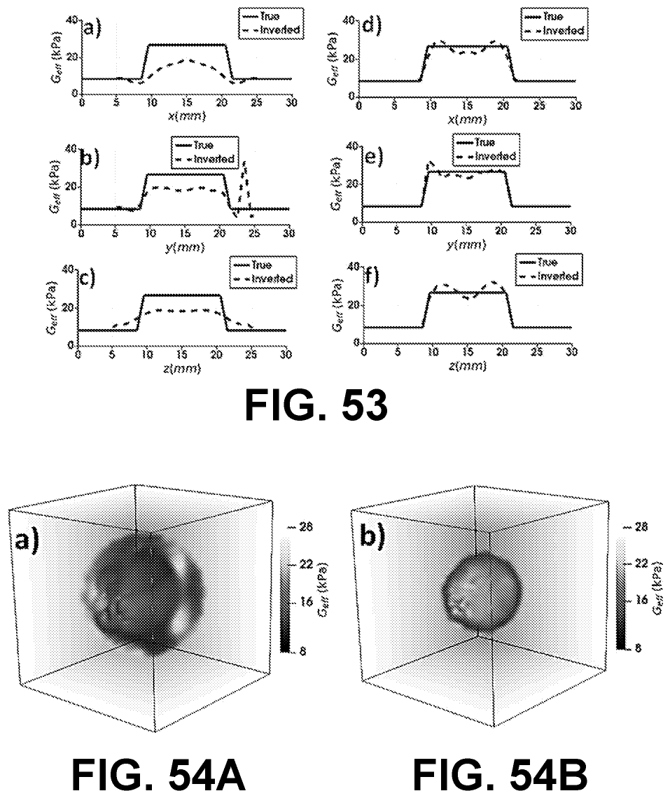

FIGS. 51A-51B, 52 and 53 show the reconstructed map for both resolutions. FIGS. 51A and 51B show the reconstructed 3D map of the effective shear modulus for the 12 mm tumor: with (a) resolution 1 (100-250 Hz), and (b) resolution 2 (100-400 Hz), and with a SNR=30 dB. FIG. 52 shows the reconstructed 2D views of the effective shear modulus for the 12 mm tumor: with (a-c) resolution 1 (100-250 Hz), and (d-f) resolution 2 (100-400 Hz) and with a SNR=30 dB. FIG. 53 shows the comparison between the reconstructed and true effective shear modulus for the 12 mm tumor along x-, y-, z-axes that passes through the center of the tumor: with (a-c) resolution 1 (100-250 Hz), and (d-f) resolution 2 (100-400 Hz) and with a SNR=30 dB. While there is a noise with SNR=30, there is a very good match between the reconstructed and the true values of the modulus.

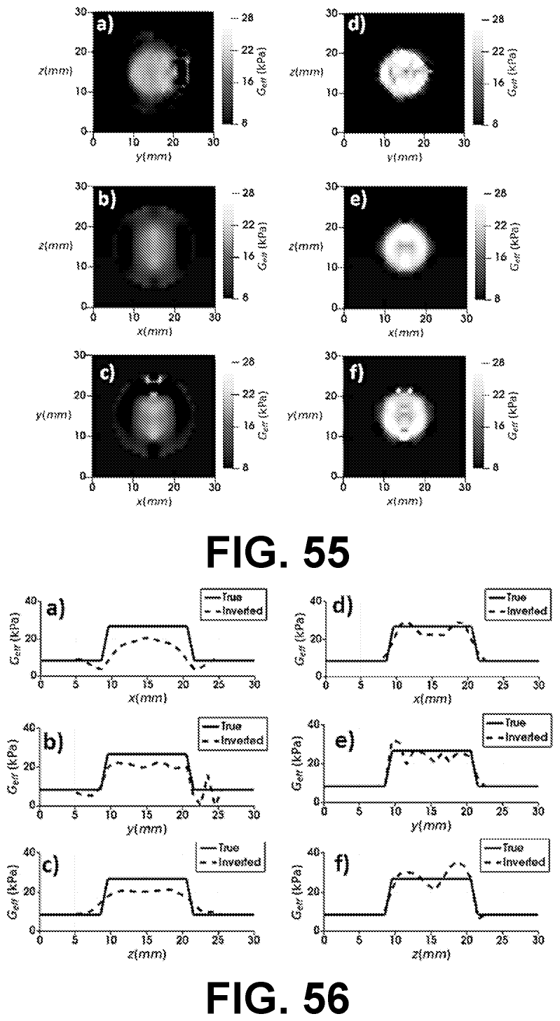

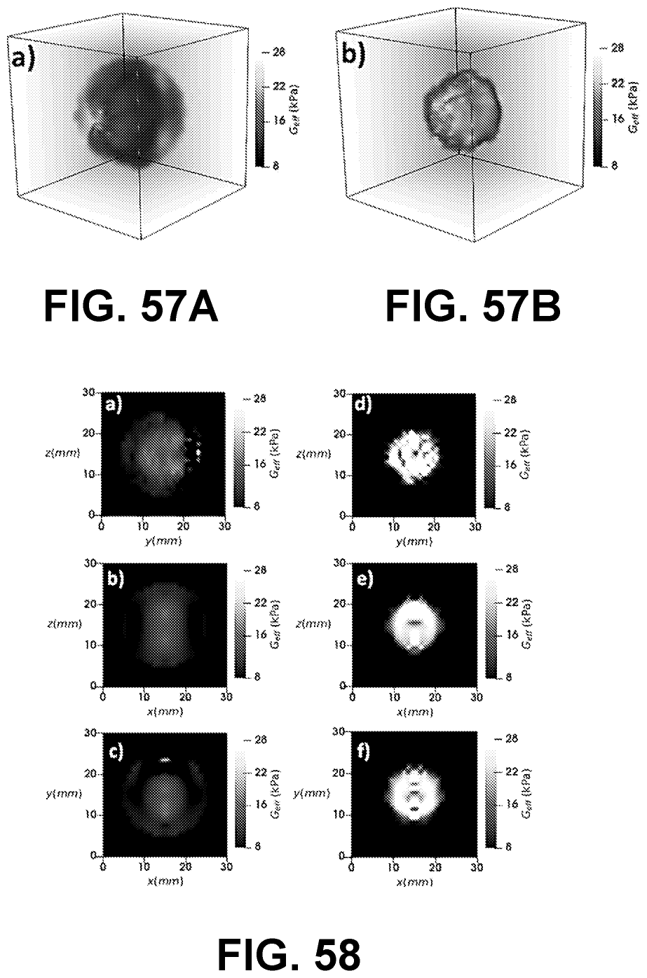

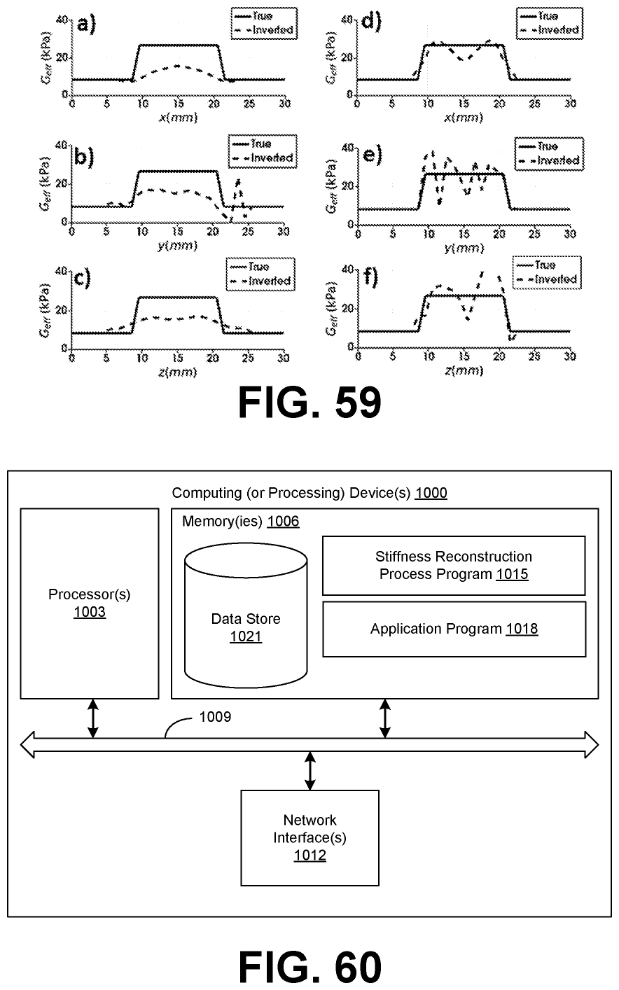

Similar conclusions can be drawn for reconstructing with a SNR=20 dB. FIGS. 54A and 54B show the reconstructed 3D map of the effective shear modulus for the 12 mm tumor: with (a) resolution 1 (100-250 Hz), and (b) resolution 2 (100-400 Hz), and with a SNR=20 dB. FIG. 55 shows the reconstructed 2D views of the effective shear modulus for the 12 mm tumor: with (a-c) resolution 1 (100-250 Hz), and (d-f) resolution 2 (100-400 Hz) and with a SNR=20 dB. FIG. 56 shows the comparison between the reconstructed and true effective shear modulus for the 12 mm tumor along x-, y-, z-axes that passes through the center of the tumor: with (a-c) resolution 1 (100-250 Hz), and (d-f) resolution 2 (100-400 Hz) and with a SNR=20 dB. The reconstructed maps for SNR=30 seem to be slightly better, which is expected. On the other hand, for the case with SNR=10, the reconstructed maps seem to have the least accuracy, although the tumor shape, size and the mechanical properties are “on average” close to the true ones. FIGS. 57A and 57B show the reconstructed 3D map of the effective shear modulus for the 12 mm tumor: with (a) resolution 1 (100-250 Hz), and (b) resolution 2 (100-400 Hz), and with a SNR=10 dB. FIG. 58 shows the reconstructed 2D views of the effective shear modulus for the 12 mm tumor: with (a-c) resolution 1 (100-250 Hz), and (d-f) resolution 2 (100-400 Hz) and with a SNR=10 dB. FIG. 59 shows the comparison between the reconstructed and true effective shear modulus for the 12 mm tumor along x-, y-, z-axes that passes through the center of the tumor: with (a-c) resolution 1 (100-250 Hz), and (d-f) resolution 2 (100-400 Hz) and with a SNR=10 dB. These all indicate that it is technically feasible to reconstruct a complete 3D map of soft tissue's mechanical properties utilizing the existing SWE measurements.

In this work, reconstructing 3D maps of soft tissues using a setup similar to conventional ultrasound imaging has been presented. Shear wave motions have been used that are synthetically generated after application of ARF and detected in a y-z plane to reconstruct the whole 3D maps of tumor-like tissue. Two tumors with different sizes were successfully reconstructed using the proposed methodology. Even in the existence of low SNR, we were able to reconstruct the tumor's geometrical and mechanical properties. In all the presented examples, we used one ARF and one plane for the reconstruction.

Multiple approaches would improve the proposed imaging framework. Using different parameterizations that show better sensitivity can provide a more robust inversion framework. By regularizing the objective functional, a more robust inversion is possible, especially at low SNR.

With reference to FIG. 60, shown is a schematic block diagram of a computing (or processing) device 1000 that can be utilized for stiffness reconstruction of soft tissues or other applications using the described techniques. In some embodiments, among others, the computing device 1000 may represent a mobile device (e.g., a smartphone, tablet, computer, etc.) or other processing device. Each computing device 1000 includes processing circuitry comprising at least one processor circuit, for example, having a processor 1003 and a memory 1006, both of which are coupled to a local interface 1009. To this end, each computing device 1000 may comprise, for example, at least one server computer or like device. The local interface 1009 may comprise, for example, a data bus with an accompanying address/control bus or other bus structure as can be appreciated.

In some embodiments, the computing (or processing) device 1000 can include one or more network interfaces 1012. The network interface 1012 may comprise, for example, a wireless transmitter, a wireless transceiver, and a wireless receiver. As discussed above, the network interface 1012 can communicate to a remote computing device using a Bluetooth protocol. As one skilled in the art can appreciate, other wireless protocols may be used in the various embodiments of the present disclosure.

Stored in the memory 1006 are both data and several components that are executable by the processor 1003. In particular, stored in the memory 1006 and executable by the processor 1003 are stiffness reconstruction process program 1015, application program 1018, and potentially other applications. Also stored in the memory 1006 may be a data store 1021 and other data. In addition, an operating system may be stored in the memory 1006 and executable by the processor 1003.

It is understood that there may be other applications that are stored in the memory 1006 and are executable by the processor 1003 as can be appreciated. Where any component discussed herein is implemented in the form of software, any one of a number of programming languages may be employed such as, for example, C, C++, C#, Objective C, Java®, JavaScript®, Perl, PHP, Visual Basic®, Python®, Ruby, Flash®, or other programming languages.

A number of software components are stored in the memory 1006 and are executable by the processor 1003. In this respect, the term “executable” means a program file that is in a form that can ultimately be run by the processor 1003. Examples of executable programs may be, for example, a compiled program that can be translated into machine code in a format that can be loaded into a random access portion of the memory 1006 and run by the processor 1003, source code that may be expressed in proper format such as object code that is capable of being loaded into a random access portion of the memory 1006 and executed by the processor 1003, or source code that may be interpreted by another executable program to generate instructions in a random access portion of the memory 1006 to be executed by the processor 1003, etc. An executable program may be stored in any portion or component of the memory 1006 including, for example, random access memory (RAM), read-only memory (ROM), hard drive, solid-state drive, USB flash drive, memory card, optical disc such as compact disc (CD) or digital versatile disc (DVD), floppy disk, magnetic tape, or other memory components.

The memory 1006 is defined herein as including both volatile and nonvolatile memory and data storage components. Volatile components are those that do not retain data values upon loss of power. Nonvolatile components are those that retain data upon a loss of power. Thus, the memory 1006 may comprise, for example, random access memory (RAM), read-only memory (ROM), hard disk drives, solid-state drives, USB flash drives, memory cards accessed via a memory card reader, floppy disks accessed via an associated floppy disk drive, optical discs accessed via an optical disc drive, magnetic tapes accessed via an appropriate tape drive, and/or other memory components, or a combination of any two or more of these memory components. In addition, the RAM may comprise, for example, static random access memory (SRAM), dynamic random access memory (DRAM), or magnetic random access memory (MRAM) and other such devices. The ROM may comprise, for example, a programmable read-only memory (PROM), an erasable programmable read-only memory (EPROM), an electrically erasable programmable read-only memory (EEPROM), or other like memory device.

Also, the processor 1003 may represent multiple processors 1003 and/or multiple processor cores and the memory 1006 may represent multiple memories 1006 that operate in parallel processing circuits, respectively. In such a case, the local interface 1009 may be an appropriate network that facilitates communication between any two of the multiple processors 1003, between any processor 1003 and any of the memories 1006, or between any two of the memories 1006, etc. The local interface 1009 may comprise additional systems designed to coordinate this communication, including, for example, performing load balancing. The processor 1003 may be of electrical or of some other available construction.

Although the stiffness reconstruction process program 1015 and the application program 1018, and other various systems described herein may be embodied in software or code executed by general purpose hardware as discussed above, as an alternative the same may also be embodied in dedicated hardware or a combination of software/general purpose hardware and dedicated hardware. If embodied in dedicated hardware, each can be implemented as a circuit or state machine that employs any one of or a combination of a number of technologies. These technologies may include, but are not limited to, discrete logic circuits having logic gates for implementing various logic functions upon an application of one or more data signals, application specific integrated circuits (ASICs) having appropriate logic gates, field-programmable gate arrays (FPGAs), or other components, etc. Such technologies are generally well known by those skilled in the art and, consequently, are not described in detail herein.

Also, any logic or application described herein, including the stiffness reconstruction process program 1015 and the application program 1018, that comprises software or code can be embodied in any non-transitory computer-readable medium for use by or in connection with an instruction execution system such as, for example, a processor 1003 in a computer system or other system. In this sense, the logic may comprise, for example, statements including instructions and declarations that can be fetched from the computer-readable medium and executed by the instruction execution system. In the context of the present disclosure, a “computer-readable medium” can be any medium that can contain, store, or maintain the logic or application described herein for use by or in connection with the instruction execution system.

The computer-readable medium can comprise any one of many physical media such as, for example, magnetic, optical, or semiconductor media. More specific examples of a suitable computer-readable medium would include, but are not limited to, magnetic tapes, magnetic floppy diskettes, magnetic hard drives, memory cards, solid-state drives, USB flash drives, or optical discs. Also, the computer-readable medium may be a random access memory (RAM) including, for example, static random access memory (SRAM) and dynamic random access memory (DRAM), or magnetic random access memory (MRAM). In addition, the computer-readable medium may be a read-only memory (ROM), a programmable read-only memory (PROM), an erasable programmable read-only memory (EPROM), an electrically erasable programmable read-only memory (EEPROM), or other type of memory device.

Further, any logic or application described herein, including the stiffness reconstruction process program 1015 and the application program 1018, may be implemented and structured in a variety of ways. For example, one or more applications described may be implemented as modules or components of a single application. For example, separate applications can be executed for the viscoelasticity estimation workflows. Further, one or more applications described herein may be executed in shared or separate computing devices or a combination thereof. For example, a plurality of the applications described herein may execute in the same computing device 1300, or in multiple computing devices in the same computing environment. Additionally, it is understood that terms such as “application,” “service,” “system,” “engine,” “module,” and so on may be interchangeable and are not intended to be limiting.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

The term “substantially” is meant to permit deviations from the descriptive term that don't negatively impact the intended purpose. Descriptive terms are implicitly understood to be modified by the word substantially, even if the term is not explicitly modified by the word substantially.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include traditional rounding according to significant figures of numerical values. In addition, the phrase “about ‘x’ to ‘y’” includes “about ‘x’ to about ‘y’”.

1. A method, comprising:
obtaining particle velocities from generated mechanical waves in tissue of a subject;
reconstructing a stiffness map of soft tissue in a region of interest (ROI) from the particle velocities; and
generating an image of the stiffness map for rendering on a user device.

2. The method of claim 1, wherein reconstructing the stiffness map of soft tissue in the ROI is based upon a cross-correlation based cost functional.

3. The method of claim 2, wherein reconstruction of the stiffness map comprises minimizing the cross-correlation-based cost functional to within a defined threshold.

4-6. (canceled)

7. The method of claim 1, wherein the reconstruction is carried out over a plurality of frequencies.

8. The method of claim 7, wherein one or more stiffness map from lower frequency data is used as an initial estimate for refined stiffness maps from broader frequency data.

9. (canceled)

10. The method of claim 1, wherein the image of the stiffness map is a three-dimensional (3D) image.

11. (canceled)

12. The method of claim 1, wherein the stiffness map comprises elastic modulus variation or viscoelasticity variation.

13. The method of claim 12, wherein a viscosity map is constructed after finalizing an elastic modulus map.

14. The method of claim 12, wherein a viscoelastic mechanical model comprises a general frequency dependent complex modulus.

15-18. (canceled)

19. The method of claim 1, wherein the ROI is determined based upon initial inspection of acoustic information.

20. A system to reconstruct stiffness of soft tissue, comprising:
an ultrasound scanner configured for shear wave elastography (SWE); and
a computing device comprising a processor and memory, the computing device configured to at least:
obtain particle velocities using the ultrasound scanner, from generated mechanical waves in tissue of a subject;
reconstruct a stiffness map of soft tissue in a region of interest (ROI) from the particle velocities; and
generate an image of the stiffness map for rendering on the computing device or a user device.

21-22. (canceled)

23. The system of claim 20, wherein reconstruction of the stiffness map comprises:
determining a back propagated misfit; and
determining a gradient based upon the back propagated misfit and a forward propagating wavefield.

24. The system of claim 23, wherein the forward propagating wavefield and the back propagated misfit are computed using full waveform modeling.

25. The system of claim 23, wherein the back propagated misfit is a modified back propagated misfit.

26-27. (canceled)

28. The system of claim 20, wherein the reconstruction is carried out directly by matching time histories.

29. (canceled)

30. The system of claim 20, wherein the image of the stiffness map is reconstructed from particle velocities on one or more lower dimensional manifold.

31-33. (canceled)

34. The system of claim 20, wherein the particle velocities are generated from acoustic radiation forces produced by the ultrasound scanner.

35. The system of claim 34, wherein multiple acoustic radiation forces are used to increase sensitivity of measurements to viscoelasticity maps.

36. The system of claim 35, wherein the multiple acoustic radiation forces are located on multiple sides of the ROI.

37. The system of claim 36, wherein push locations are based upon initial inspection of the acoustic information.

38. (canceled)
